(19) United States
(12) Patent Application Publication
(10) Pub. No.: US 2026/0061107 A1
(43) Pub. Date: Mar. 5, 2026

(54) Apparatus, System, and Method for Extracorporeal Cooling of Whole Blood

(71) Applicant: Alfredo R. Zarate, Bethesda, MD (US)

(72) Inventor: Alfredo R. Zarate, Bethesda, MD (US)

(21) Appl. No.: 19/304,265

(22) Filed: Aug. 19, 2025

Related U.S. Application Data
(60) Provisional application No. 63/689,494, filed on Aug. 30, 2024.

(51) Int. Cl. A61M 1/36; A61M 60/113; A61M 60/279; A61M 60/36

(52) U.S. Cl. CPC A61M 1/369; A61M 1/3623; A61M 1/3626; A61M 1/3638; A61M 1/3659; A61M 60/113; A61M 60/279; A61M 60/36; A61M 2202/0413; A61M 2205/18; A61M 2205/3334; A61M 2205/3368; A61M 2205/3606; A61M 2205/366; A61M 2205/502

(57) ABSTRACT

Cardiac arrest (CA) and traumatic brain injury (TBI) are critical medical events associated with high mortality and long-term disability, primarily due to ischemia and subsequent inflammation. Targeted temperature management (TTM), particularly hypothermia, has proven beneficial in reducing cell death, preserving brain function, and mitigating inflammation following these events. Conventional TTM methods are limited by delayed temperature reduction, poor temperature maintenance, and risks such as thromboembolism. The present invention addresses these challenges by introducing novel devices, apparatus, and systems for extracorporeal whole blood cooling, enabling rapid, controlled induction and maintenance of therapeutic hypothermia. By efficiently cooling the patient's whole blood volume outside the body, the method of the present invention offers a safer, more effective alternative to traditional methods, significantly improving patient outcomes after events such as CA and TBI. The present invention represents a promising advancement in emergency and critical care, enhancing survival and neurological recovery in severely ill patients.

### FIELD OF THE INVENTION

The present invention generally relates to medical sciences and devices for temperature treatment and modulation in clinical care settings. More specifically, it relates to apparatuses, systems, devices, and methods for use thereof to induce hypothermia by extracorporeal cooling of whole blood.

### INCORPORATION BY REFERENCE

All publications, patents, and patent applications mentioned in this specification are herein incorporated by reference in their entirety to the same extent as if each publication or patent application was specifically and individually indicated to be incorporated by reference.

### BACKGROUND OF THE INVENTION

In the United States, more than 356000 cases of out-of-hospital-cardiac arrest (OOHCA) and over 290000 cases of adult cases of in-hospital-cardiac-arrest (IHCA) occur per year. Sudden cardiac arrest (CA) accounts for 15-20% of all natural deaths in adults in the USA and Western Europe, and up to 50% of all cardiovascular deaths. Most patients that survive CA are in a coma immediately after the recovery of circulation. One of three experience delayed recovery of consciousness, 23% remain in coma one week and between 15-30% of all patients with a good prognosis regain consciousness between 48 hours and 10-12 days after the suspension of sedation. Some patients experience post-resuscitation syndrome caused by ischemia of many organs and the inflammatory process that follows it, characterized by neurological deficits, multiple organ dysfunction, infections, and high mortality, and about 33% develop hyperthermia (>37.5° C.) 24-48 hours after a cardiac arrest.

In 2011, 7.6% of OHCA and 18% of IHCA survived hospital discharge, and half of the patients who survived had severe neurological deficits. In a study of IHCA, 67% achieved return of spontaneous circulation between 3-13 min, 33% between 14-30 min, and 22% of all patients survived to hospital discharge. The one with return of circulation after one minute of CPR had a 22% survival, but <1% after 39 minutes of CPR. Many of the survivors died a few months after discharge, and irreversible brain injury was the most common cause of death; others lived in a vegetative state with severe neurological disabilities. In the same study, TBI occurs in 50 million people annually worldwide and costs the global economy $400 billion. In the UK, it is the most common cause of death and disability under the age of 40. Severe TBI, a Glasgow Coma Scale score of 3-8, has a 40% mortality and causes major psychosocial deficits in 60% of cases.

In cardiac arrest (CA) and traumatic brain injury (TBI), brain ischemia is a major cause of neurological damage but in TBI, the damage is caused by additional modes of injury; physical impact (extrinsic compression from mass lesion or contusion), diffuse axonal injury (shearing of axons), inflammation, cortical spreading depression (CSD), and microvascular thrombosis in differing proportions with resultant different clinical presentation and outcomes. Additionally, research in rats has shown that TBI can alter genomic transcripts, microRNA responses, and protein synthesis.

Brain cell damage develops in a few seconds or minutes of ischemia, and the duration of ischemia is a critical factor in a patient's survival and residual disabilities. Ischemia decreases the cells' metabolic rate and oxygen consumption, and ATP levels. It causes anoxic depolarization, also called cell membrane ion pump failure, which includes loss of potassium and glutamate, increase in calcium, mitochondrial depolarization, increased extracellular excitatory amino acids glutamate and aspartate that cause excitatory neurotoxicity (cell death) and swelling of neurons and dendrites, also called cytotoxic edema. It is reflected by a loss of membrane electrical potential that marks the start of irreversible injury, which occurs a few seconds or minutes later with terminal depolarization. In survivable brain ischemia, cell depolarization is localized and reversible, whereas in non-survivable ischemia, it spreads to other cells and is not reversible. Ischemia also stimulates an inflammatory response with an increase of immune cells, release of pro-inflammatory cytokines, and excessive generation of reactive oxygen and nitrogen species that overwhelm the capacity of the neurons biological antioxidants (glutathione, catalase and glutathione peroxidase, etc.) causing oxidative stress, which can lead to cells death via increased apoptosis or cytotoxicity, inactivation of antiapoptotic mechanism, increased release of cellular proteases such as calpain which cleaves cytoplasmic and nuclear substrates, leading to apoptosis and sceptrin (structural protein that maintains the stability and structure of the cell membrane and the shape of a cell), both appearing along morphological evidence of fatal cell injury and an increase of neuron-specific-enolase in plasma, a biomarker of neuronal death. These cellular changes have been described as a “two-hit model” in which the primary events are caused by ischemia and the secondary injury by inflammation. Hyperthermia increases oxygen consumption, the release of neurotransmitters and proteases, the production of oxygen free radicals, and neuronal death.

Today, TTM is the only strategy known to be able to provide effective neuroprotection post-CA, especially if started immediately after the event, and it is standard treatment in adult survivors of OHC with an initial shockable rhythm who remain unresponsive after return of spontaneous circulation. It is a standard therapy recommended by the American Heart Association, the European Resuscitation Council, and the International Liaison Committee on Resuscitation (ILCOR). TTM is also widely used to treat TBI and selected patients post-brain surgery for the same reasons.

Historically, therapeutic hypothermia or TTM has been used in various forms, with documented use dating back to ancient times. In modern medicine, clinical studies have demonstrated that TTM significantly improves neurological outcomes and survival rates in comatose patients following resuscitation after cardiac arrest. The neuroprotective benefits stem from a reduction in cerebral metabolic rate, inflammation, excitotoxicity, and free radical formation associated with reperfusion injury. The strategic modulation of body temperature can significantly influence cellular metabolism, inflammatory pathways, and neuronal recovery, thereby altering the trajectory of patient outcomes in a variety of high-risk clinical scenarios.

TTM leads to neuroprotection by reducing all the above processes; it allows cells to tolerate longer periods of ischemia without irreversible damage, attenuates the neurological damage induced by the inflammatory response and the rise of intracranial pressure caused by brain edema, prevents hyperthermia, and improves neurological outcomes. For every 1° C. fall in temperature, the cerebral metabolism falls by 8%, which in turn leads to a decrease in cerebral metabolic requirement of oxygen (CMRO₂) and cerebral blood flow. Moreover, it modulates the inflammatory and apoptotic signaling pathways, favoring trophic and anti-apoptotic protein synthesis, reduction of excitatory neurotransmitters, and inflammatory response.

Cardiac arrest, characterized by the abrupt cessation of cardiac mechanical activity, whether occurring inside or outside a hospital setting, often leads to significant neurological injury due to cerebral hypoxia. Among the interventions aimed at minimizing post-anoxic brain injury, TTM is especially vital in patients with spontaneous circulation following cardiac arrest, where brain injury due to global cerebral ischemia remains a leading cause of mortality and long-term disability. TTM has emerged as a clinical strategy to mitigate neurologic injury by controlling the core body temperature of patients, and it is the only neuroprotective therapy currently recommended by international guidelines by professional societies including the American Heart Association and the European Resuscitation Council, where the controlled reduction and maintenance of a patient's core body temperature mitigates reperfusion injury and improves neurological outcomes. This is because cooling the body in a controlled manner has been shown to reduce neuronal injury, stabilize the blood-brain barrier, and improve neurologic outcomes.

Beyond cardiac arrest, TTM has proven utility in other neurocritical conditions, such as traumatic brain injury (TBI), postoperative care following brain surgery, and situations where there is a risk of increased intracranial pressure (ICP). In these settings, temperature modulation can help mitigate secondary brain injury by reducing cerebral metabolic demand, attenuating excitotoxicity, and limiting edema formation. Similarly, in neonates with hypoxic-ischemic encephalopathy, early initiation of TTM has been shown to reduce mortality and improve neurodevelopmental outcomes by interrupting the cascade of injury following perinatal asphyxia.

In extracorporeal membrane oxygenation (ECMO) support for cardiogenic shock or acute respiratory distress syndrome (ARDS), TTM serves a dual role: it not only contributes to neuroprotection in patients with concomitant cerebral hypoxia but also enhances overall metabolic control, helping to optimize oxygen utilization and reduce systemic inflammatory responses. Given the complex interplay between temperature, perfusion, and organ recovery in such critical care scenarios, the need for precise and responsive TTM strategies is more pressing than ever.

Therefore, the implementation of TTM across a range of indications, including post-cardiac arrest care, traumatic and hypoxic brain injuries, neurosurgical recovery, and ECMO support, underscores its central role in improving survival and neurological function. As clinical protocols continue to evolve, the integration of reliable, adaptive TTM systems becomes essential for delivering high-quality, personalized care in intensive care settings.

Conventionally, TTM involves the controlled reduction and maintenance of a patient's core body temperature, typically within the range of 32° C. to 36° C., over a specific period, usually 24 hours or longer. Despite growing adoption, existing TTM protocols vary significantly across institutions, including differences in cooling methods, target temperature ranges (typically 32° C. to 36° C. as aforementioned), and duration of temperature maintenance. Clinical and research findings have yielded ambiguous results when comparing target temperatures of 33° C. and 36° C., coupled with variability in TTM execution, such as inconsistent induction times, maintenance periods, rewarming protocols, and methods of temperature monitoring, which has led to widespread clinical uncertainty. The comparative effectiveness of various target temperatures, the optimal methods and timing for inducing hypothermia, and the duration of cooling and rewarming remain active areas of clinical investigation. In many institutions, this has resulted in either the abandonment of TTM or the application of inconsistent protocols, undermining potential clinical benefits. Additionally, complications such as shivering, electrolyte imbalance, arrhythmias, and coagulopathies pose challenges to consistent and safe implementation. Therefore, there is a need for systems and methods that can rapidly, safely, and precisely implement TTM protocols, minimize variability in delivery, and account for patient-specific risk factors and physiologic responses.

There are surface cooling, blankets or pads with circulating cold air or water, and endovascular catheters with ice-saline-filled balloons. Extracorporeal cooling of blood was tried over a decade ago but abandoned because of cost and logistical difficulties. Current and conventional systems and methods of TTM include surface cooling with ice packs or cooling blankets, internal cooling with catheter-based systems or cold fluid infusions, and specialized systems such as heat-exchange pads and intravascular cooling catheters. Despite technological advancements, existing devices still face challenges related to inconsistent temperature maintenance, overshooting target temperatures, user complexity, patient discomfort, delayed induction, and risks of thermal injury.

Potential complications caused by hypothermia or TTM include shivering increases cerebral metabolism, increased cerebral metabolism, increased cerebral blood flow, and increased intracranial pressure. It starts at 35.5° C. and stops at 33.5° C. Prolongation of the PR interval widened QRS and QTc, tachycardia in early phases, and bradycardia with lower cardiac output (25%-40%) with mild to moderate hypothermia. At a temperature of 32° C. a heart rate of 40/min is normal. The risk of arrhythmia is high at core temperature above 30° C. Atrial fibrillation (AF) is most common in moderate to deep hypothermia, ventricular tachycardia or ventricular fibrillation may occur with <28° C., hypokalemia, and hypomagnesemia favor the development of ventricular arrhythmias. The catecholamines released in the early stages cause arterial vasoconstriction and hypertension. Very low temperatures of long duration, i.e., >24 hours, can cause hypotension, decreased cardiac output/bradycardia because of decreased catecholamine responsiveness. Arterial vasoconstriction increases the central venous pressure because of increased preload (venous return) and decreases organ perfusion, which causes metabolic acidosis with increased lactic acid and hyperglycemia because of reduced insulin output. Bleeding due to alteration of the coagulation parameters and thrombocytopenia may occur. Infections occur in up to 70% of cases, and their incidence is almost twofold higher than with normothermia. Pneumonia is the most common (85%) and is associated with increased mortality. The hemoglobin dissociation curve shifts to the left, hindering the release of oxygen to the tissues. Loss of blood volume and intracellular shifts lead to hypokalemia and hyponatremia. During rewarming, this is reversed. It decreases gastrointestinal motility. Serum amylase and liver enzymes increase.

Side effects caused by the existing methods to deliver TTM, such as by surface cooling methods, include overshooting the lower temperatures and inducing rapid or excessive re-warming temperatures that may increase cerebral edema and intracranial pressure. Endovascular cooling catheters may cause fatal thromboembolism because platelets impact or friction on the looped catheter, and the turbulence or flow disturbance that occurs when blood impacts and goes around a catheter contributes to activating or destroying platelets, attaching them to the catheter, and forming clots. The device also shares the risks of other extracorporeal circulation equipment, i.e., dialysis, cardio-pulmonary bypass, including hypotension, bleeding, clotting of tubes, adverse reactions to materials or heparin, pyrogen reactions, electro-mechanical/software failure, air embolism, hemolysis, and blood loss from ruptured or misfitted tubes.

For example, U.S. Pat. No. 11,374,231B2 provides a device for extracorporeal treatment of plasma using ultraviolet light (UVL) to inactivate pathogens. This device includes a UVL unit and components to cool and heat plasma and can be used to maintain normothermia (˜37° C.) by ceasing UVL and heat exchange operations. However, this previously patented device is limited in scope to plasma treatment and lacks utility in delivering controlled hypothermia for therapeutic applications. Furthermore, the prior invention depends on a UVL module not required in targeted temperature management (TTM) or therapeutic hypothermia. There remains an unmet clinical need for a dedicated extracorporeal system capable of whole blood cooling to induce hypothermia and provide precision thermal control in various critical care scenarios.

Even though TTM has been found effective after CA in patients and animals, several clinical studies have not shown clear benefits over normothermia. Likely this is the case because the outcomes depend on multiple factors including, length of time and extends of the ischemia, whether is complete or partial, the adequacy of CPR (perfusion), the use of neuroprotective strategies, ineffective methods to induce, maintain and rewarm, persistence of cerebral hypoperfusion, the presence and severity of co-morbidities, and most of the clinical studies performed did not have, similar duration of ischemia or number and severity of co-morbidities. IHCA patients are older than OHCA, mean age of 66, 80% have a non-shockable rhythm, i.e., asystole or pulseless electrical activity, and the cause of the CA is 50-60% cardiac and 15-40% respiratory. There is a 2.5-fold difference in survival to hospital discharge between OHCA and IHCA. Some studies have been underpowered. And in almost all the studies, many patients had either a prolonged phase of ischemia, i.e., OHCA, or a delayed start of TTM. Nevertheless, TTM is still indicated in all patients with CA because, at present, there is no good method to identify the presence and magnitude of brain damage early after a CA. It takes 3 to 7 days to clinically identify reversible or irreversible neurological damage. There is irreversible damage in absence of pupil and corneal reflexes after initial 24 hours, and bilateral absence of evoked potentials which is more specific after 72 hours, elevated blood level of enolase at 72 hours, a Glasgow Coma Scale <2, presence of myoclonus, EEG without reactivity in the first 12 hours, absent auditory reactivity after 48 hours, and an MRI scan with diffusion alterations in the first week. Laboratory and clinical studies have demonstrated that when TTM is applied as soon as possible after CPR, it results in better neurological outcomes. To improve the outcomes, early and effective CPR, TTM, and treatments to improve the cell's function are needed. Recently, Ghrelin, “the hunger hormone,” has improved function and histology in these patients. TTM is also widely used in TBI and neurosurgery, even though many studies have not shown that it contributes to changing outcomes. Similarly to post-CA, at present, there are no methods sensitive enough to assess the severity and extent of the neurological lesions present in a given patient. Thus, it is not possible to know a priori which patient may benefit from its use, and many clinical trials did not have enough power, homogeneity of trial design, and inclusion criteria. Nevertheless, “The scientific basis for temperature control is strong . . . the challenge is to identify patients who should and should not be treated with TTM”, and the same should apply to post-TBI.

Considering the above, there remains a need for improved TTM systems that provide faster induction of target temperature along with precise temperature maintenance with minimal deviation, while reducing the risk of complications such as skin injury or hemodynamic instability, but have ease of application across diverse clinical environments, including pre-hospital, emergency department, and intensive care settings, and a capability and capacity for integration with modern monitoring systems including automation and artificial intelligence based hands-free operation. Correspondingly, there exists a need for an improved system or method for implementing TTM that standardizes temperature modulation phases, minimizes complications, enhances the consistency, safety of temperature regulation, and optimizes outcomes with minimal resource burden. Also, there remains an unmet clinical need for a dedicated extracorporeal system capable of whole blood cooling to induce hypothermia and provide precision thermal control in various critical care scenarios.

The present invention addresses this need and problem by providing apparatuses, systems, devices, and methods for use thereof to induce hypothermia by delivering targeted temperature management (TTM) through extracorporeal cooling of whole blood. The device, apparatus, or system of the present invention fulfills a medical need, it provides more effective therapy with fewer side effects than currently used methods, resolves a logistical problem (the need for many therapists), and decreases the cost of care.

### SUMMARY OF THE INVENTION

The following listing of embodiments is a non-limiting statement of various aspects of the invention. Other aspects and variations will be evident in light of the entire disclosure.

The present invention provides a new device, system, apparatus, and method of use thereof in methods of treatment and/or prevention in patients requiring the induction of hypothermia and delivering targeted temperature management (TTM) by extracorporeally cooling whole blood. This device system, apparatus, and method of use thereof is to be used to treat and/or prevent further complications in patients with spontaneous beating hearts post cardiac arrest, traumatic brain injury, after brain surgery with or before a rise in intracranial pressure, in hypoxic brain injury in neonates, and with extracorporeal membrane oxygenator (ECMO) for cardiogenic shock, and acute respiratory distress syndrome (ARDS).

An aspect of the present invention provides a device or system or apparatus to deliver targeted temperature management to a patient, comprising: a first whole blood circulation equipment; a circuit of tubes; an extracorporeal cooling unit; and a second whole blood circulation equipment, wherein the device or system or apparatus delivers targeted temperature management by cooling circulating whole blood from a patient extracorporeally to a target temperature as the whole blood circulates through the device or system or apparatus to obtain cooled whole blood at the target temperature that is circulated back into the patient, wherein the targeted temperature management includes cooling, maintaining, and increasing the core temperature of the patient to the target temperature by changing the temperature of the whole blood of the patient being circulated through the device or system or apparatus, wherein the first whole blood circulation equipment is used to draw whole blood from a patient continuously, wherein the first whole blood circulation equipment is selected from a group consisting of a first single lumen catheter, a first metal needle, and a first lumen of a double lumen catheter, wherein the whole blood drawn from the patient circulates at a flow rate in a range of between 50 and 200 milliliters per minute (ml/min) through the circuit of tubes, wherein the extracorporeal cooling unit is selected from a group consisting of one or more heat-exchangers, one or more coiled tubes, and one or more containers containing one or more cooling liquids or cooling fluids or coolants, wherein the cooled whole blood is returned to the patient via the second whole blood circulation equipment, wherein the second whole blood circulation equipment is selected from a group consisting of a second single lumen catheter, a second metal needle, and a second lumen of the double lumen catheter, wherein the first single lumen catheter, the first metal needle, or the first lumen of the double lumen catheter is inserted into a first vein of the patient, and the second single lumen catheter, the second metal needle, or the second lumen of the double lumen catheter is inserted into a second vein of the patient, wherein the first vein and the second vein are different veins of the patient, wherein the first lumen of the double lumen catheter is arterial and referred to as an arterial lumen, and the second lumen of the double lumen catheter is venous and referred to as a venous lumen, wherein the target temperature is a desired low core body temperature for the patient, and it is a pre-determined temperature set by an operator of the device or system or apparatus in a mode selected from a group consisting of manual mode, automatic mode, and semi-automatic mode, and wherein the desired low core temperature is selected from a range of temperatures between 32° C. and 36° C.

An aspect of the present invention provides a device or system or apparatus to deliver targeted temperature management to a patient, comprising: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10) for cooling liquids or cooling fluids or coolants; a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20), wherein the circuit of tubes with segments transports the whole blood from and back to the patient or a container during in vitro testing after the whole blood circulates once fully through the device or system or apparatus, wherein the first tube (2), the second tube (3), the third tube (4), the fourth tube (5), the fifth tube (6) have an internal diameter in a range of between 1.5 millimeters (mm) and 2 mm for patients who are adults, and in a range of between 0.3 mm and 0.4 mm for patients who are children, wherein the first tube (2), the second tube (3), the third tube (4), the fourth tube (5), the fifth tube (6) have a wall thickness of 1 mm, wherein the first tube (2), the second tube (3), the third tube (4), the fourth tube (5), the fifth tube (6) have a length in a range of between 10 feet (ft) and 14 ft for patients who are adults, and in a range of between 5 ft and 9 ft for patients who are children, wherein the first tube (2), the second tube (3), the third tube (4), the fourth tube (5), the fifth tube (6) are made of material selected from a group consisting of Poly Vinyl Chloride (PVC), Polydimethylsiloxane (PDMS), Dow Corning and combinations thereof, wherein the fifth tube (6) that transports the whole blood back to the patient (1) has the air and clot trap (7) on or in it, wherein the air and clot trap (7) is 7 centimeters (cm) in length and has a diameter of 1.5 cm, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling liquid or cooling fluid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling liquid or cooling fluid or coolant flow rate sensor to regulate the cooling and heat exchange in the cooling unit (9) between the whole blood and the cooling liquid or cooling fluid or coolant, wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19) and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core temperature for the patient, wherein the low core temperature is selected from a range of temperatures between 32° C. and 36° C., and wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS.

An aspect of the present invention provides a method for delivering targeted temperature management to a patient, the method comprising the steps of: (a) providing a device or system or apparatus to deliver targeted temperature management to a patient, the device or system or apparatus comprising: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10); a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20); (b) inserting the first tube (2) inside a first central vein of the patient to draw the whole blood from the patient and transport it to the cooling unit (9) inside the device or system or apparatus; (c) inserting the fifth tube (6) inside a second central vein of the patient to return the whole blood from the device or system or apparatus to the patient (1) after it cools down to a target temperature; (d) administering Heparin immediately before or with the next step; (e) starting the device or system or apparatus to deliver targeted temperature management to the patient and commencing an induction phase of the target temperature management; (f) changing to and commencing a maintenance phase of the target temperature management; (g) changing to and commencing a rewarming phase of the target temperature management; and (h) stopping the device or system or apparatus, wherein the circuit of tubes with segments transports the whole blood from and back to the patient (1) or a container during in vitro testing after the whole blood circulates once fully through the device or system or apparatus, wherein the first tube (2) is referred to as the arterial segment of the circuit of tubes that transports the whole blood from the patient (1) to the cooling unit (9) inside the device or system or apparatus, wherein the fifth tube (6) is referred to as the venous segment of the circuit of tubes that transports and returns the whole blood after cooling the whole blood to the target temperature in the device or system or apparatus to the patient (1), wherein the fifth tube (6) that transports the whole blood back to the patient (1) has the air and clot trap (7) on or in it, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling fluid or cooling liquid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling fluid or coolant flow rate sensor to regulate the cooling and heat exchange in the cooling unit (9) between the whole blood and the cooling liquid or cooling fluid or coolant; wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core body temperature for the patient, wherein the low core temperature is selected from a range of temperatures between 32° C. and 36° C., wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS, wherein the starting the device or system or apparatus to deliver targeted temperature management to the patient is done in a period in a range of between immediately after an event or trauma when spontaneous heartbeats return to minimize brain damage caused by ischemia, and 5 hours after the event or trauma, to prevent post resuscitation worsening of neurological damage triggered by the inflammatory response, and cerebral edema, wherein the core body temperature of the patient is decreased in the induction phase by the device or system or apparatus at a rate of temperature decrease measured in degrees Centigrade (° C.) per hour, which is in a range of between 2° C. to 2.5° C. per hour and 4° C. to 5° C. per hour, wherein the core body temperature of the patient is increased in the rewarming phase to 37° C. by the device or system or apparatus at a rate of temperature increase measured in ° C. per hour, which is in a range of between 0.25° C. per hour and 0.5° C. per hour in patients post a cardiac arrest event, and in a range of between 0.1° C. per hour and 0.25° C. per hour in patients post a traumatic brain injury event, wherein the targeted temperature management is done for a time in a range of between 24 hours and 120 hours for the patient from the starting in step (e) to the stopping in step (h), wherein the method for delivering targeted temperature management is operated in a mode selected from a group consisting of automatic mode, semi-automatic mode, and manual mode, each mode requiring trained personnel of varying intensity, with increasing training and expertise, progressing from the automatic mode to the semi-automatic mode and then to the manual mode.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention and are incorporated in and constitute a part of the present invention and, together with the description, serve to explain the principle of the invention.

In the drawings,

FIG. 1 illustrates an embodiment of the present invention and a representative illustration of a device, apparatus, or system based on the present invention.

FIG. 2 illustrates an alternate embodiment of the present invention and a representative illustration of a device, apparatus, or system based on the present invention.

FIG. 3 illustrates an alternate embodiment of the present invention and a representative illustration of a device, apparatus, or system based on the present invention.

FIG. 4 illustrates an alternate embodiment of the present invention and a representative illustration of a device, apparatus, or system based on the present invention.

FIG. 5 illustrates an alternate embodiment of the present invention and a representative illustration of a device, apparatus, or system based on the present invention.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as a basis for teaching one skilled in the art to variously practice the present invention.

All illustrations of the drawings are to describe selected versions of the present invention and are not intended to limit the scope of the present invention.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

Although any methods and materials similar or equivalent to those described herein can be used in the practice of the present invention, the exemplary methods, devices, and materials are described herein. For the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

As used herein, the term “about” refers to a rough estimate of the number or amount of the quantity referred to and is in the vicinity of the actual number or figure immediately following said term, where the actual number or figure or amount could be slightly higher or lower.

Cardiac arrest (CA) and traumatic brain injury (TBI) are common and severe medical events that frequently lead to high mortality rates or long-term disability among survivors. The outcome of CA is highly dependent on the duration of ischemia, the extent of tissue necrosis, and the severity of post-ischemic inflammation. Similarly, TBI outcomes are influenced by the degree of ischemia and inflammation following the initial injury.

Hypothermia is defined as a core temperature of <36° C., mild, moderate, and deep hypothermia range between 33-36° C., 28-32° C. and <28° C. The normal oral cavity temperature is 36.8±0.4° C. with diurnal variations of 0.5° C. The rectal temperature is 0.4° C. higher and reflects the core temperature, the lower esophagus is the most accurate noninvasive measurement, and the measured via a pulmonary artery catheter is the most accurate.

Targeted temperature management (TTM), also known as therapeutic hypothermia or controlled normothermia, has emerged as a critical intervention in modern critical care, particularly in the context of neurologic and cardiovascular emergencies. TTM is a medical treatment aimed at minimizing neurological damage following cardiac arrest or other ischemic brain injuries. TTM involves cooling the patient's core body temperature to a specific target, maintaining it for a fixed duration, and then gradually rewarming the patient. TTM consists of modification of the body temperature for therapeutic reasons. It has an induction phase to reach a targeted temperature, usually 1-2 hours, a maintenance phase to maintain the temperature usually one to a few days, and a rewarming phase to gradually increase the temperature to a chosen target usually one to a few days.

Clinical research has established that therapeutic hypothermia or TTM, can mitigate neuronal cell death, preserve neurological function, and suppress inflammatory responses post-injury. TTM has shown promise in preventing the spontaneous hyperthermia often observed within 48 hours following CA and has been associated with reduced mortality and improved neurological outcomes in both CA and TBI patients.

As described before, conventional and currently available methods for inducing TTM include external applications such as ice-water-soaked blankets, mattresses with circulating cold water or air, and invasive approaches such as endovascular cooling catheters cooled by circulating water. These techniques suffer from multiple limitations, including inability to achieve target core temperatures rapidly, lack of sustained temperature control over extended durations, i.e., over many hours or days, and risk of severe and fatal complications such as thromboembolism associated with invasive endovascular catheters.

The currently used endovascular catheters take longer to lower the core temperature because they only cool blood that is in contact with the walls of the “cooled” catheter. The blood does not go through or inside the catheter; it touches the outside of the cooled catheter inserted into the vein. The endovascular catheters tend to cause thromboembolism, in many cases, is fatal. Further, the external devices used conventionally only cool the skin, while the conventional endovascular devices cool blood that flows in a large vein around the needle. Further, the cardiac bypass machine or heartland machine (HLM) draws blood from a patient, oxygenates it, and returns it to the patient with assistance from blood pumps. The HLM also has a blood heating component because the blood temperature decreases while circulating extracorporeally and through the oxygenator, and it is used only during surgery.

The present invention addresses the limitations and need in the art and provides apparatuses, systems, devices, and methods for use thereof to induce hypothermia by delivering targeted temperature management (TTM) through extracorporeal cooling of whole blood. The present invention addresses the shortcomings of current TTM methodologies by providing a system and method capable of achieving rapid cooling to a target core body temperature and maintaining it consistently over a prolonged therapeutic period, with minimal invasiveness and reduced risk of complications. The device of the present invention cools the blood faster because the blood is in contact with a larger cooled surface area (the walls of the several tubes inside the cooling unit or heat exchangers, as disclosed in the present invention).

Further, the device of the present invention cools and rewarms the whole blood from a patient, not to oxygenate it, but it is used to cool and rewarm the blood when in use for 1-4 days in patients after surviving a cardiac arrest or after a traumatic brain injury or a similar primary event. The HLM described above works at higher blood flow, up to 6 L, because the goal is to oxygenate the entire all the circulating blood and perfuse the entire body. In contrast, the device of the present invention circulates a small volume of whole blood because it cools the entire volume by cooling small volumes in aliquots by passing it through its cooling unit. Further, the device of the present invention has a software or program that allows semi-automatic or automatic control or control by voice command.

As discussed before, a previous U.S. Pat. No. 11,374,231 discloses a device for extracorporeal treatment of plasma using ultraviolet light (UVL) to inactivate pathogens. This device includes a UVL unit and components to cool and heat plasma, and can be used to maintain normothermia (˜37° C.) by ceasing UVL and heat exchange operations. However, the previously patented device is limited in scope to plasma treatment and lacks utility in delivering controlled hypothermia for therapeutic applications. Furthermore, the prior invention depends on a UVL module, not required in TTM or therapeutic hypothermia. Thus, there remains an unmet clinical need for a dedicated extracorporeal system capable of whole blood cooling to induce hypothermia and provide precision thermal control in various critical care scenarios. To that end, the present invention discloses a novel extracorporeal device designed to induce hypothermia and deliver TTM by cooling whole blood. And, unlike the device described in U.S. Pat. No. 11,374,231, the system and method of the present invention does not include a UVL module and is not intended for plasma treatment. Instead, it directly circulates and cools whole blood extracorporeally, providing controlled, adjustable thermal modulation. The apparatus, system, and method of the present invention provides an extracorporeal circuit compatible with whole blood, a precision cooling unit that rapidly and safely lowers blood temperature, integrated control systems for target temperature monitoring and regulation, and optional warming modules for controlled rewarming phases. The present invention is distinct and unique in that it treats whole blood rather than plasma, omits UVL components entirely, and is specifically engineered for therapeutic hypothermia and temperature regulation.

The present invention is intended for use in patients with spontaneous circulation following cardiac arrest (CA), traumatic brain injury (TBI), hypoxic-ischemic brain injury in neonates, after neurosurgical procedures when elevated intracranial pressure is anticipated, and in conjunction with extracorporeal membrane oxygenation (ECMO) for managing cardiogenic shock and acute respiratory distress syndrome (ARDS).

The device, apparatus, system, and method of the present invention addresses the need in the art for a solution to the problems with the current and conventional means for TTM that cool the body surface or the blood of patients with the aforementioned afflictions, by advantageously cooling the body of the patient faster, maintaining a constant targeted body temperature for up to a few days, gradually increasing the temperature back to normal, and causing less side effects than the current methods, especially thromboembolism which is of common occurrence with existing endovascular devices. Thus, the present invention provides the only available device to provide TTM (cooling, maintaining, and increasing the core temperature) to patients' post-cardiac arrest (CA), traumatic brain injury (TBI), and post brain surgery by changing the temperature of the patient's blood while circulating extracorporeally through a cooling unit.

Further, the device, apparatus, system, and method of the present invention by faster induction of hypothermia, the device will decrease further damage to cells caused by ischemia, inflammation, or cerebral edema; by maintaining a low temperature for 24-96 hours, it will prevent and avoid the cells damage caused by hyperthermia which occurs in 33% of post cardiac arrest patients.

The device, apparatus, system, and method of the present invention have been designed to deliver TTM by cooling circulating blood extracorporeally. The present invention can be used in patients with spontaneous beating hearts after CA, TBI, after brain surgery with or before a rise in intracranial pressure, in hypoxic brain injury in neonates, and with extracorporeal membrane oxygenator (ECMO) for cardiogenic shock and acute respiratory distress syndrome (ARDS). In the device, apparatus, system, and method of the present invention, whole blood that is continuously drawn from a patient via a single lumen catheter or metal needle or one lumen of a double lumen catheter (or arterial lumen), circulate at a flow rate of between 50-200 milliliters per minute (ml/min), through a circuit of tubes and an extracorporeal cooling unit that could be a heat exchanger or a container with a cooling fluid and the cooled blood is returned to the patient via another single lumen catheter or metal needle, both inserted in a different vein, or the other lumen of a double lumen catheter (or venous lumen).

The device, apparatus, system, and method of the present invention additionally have one or more peristaltic pumps, various dials to change the flow rates and temperatures, a digital display to monitor and observe the flow rates and temperatures, various alarms for air in blood, and can be operated manually or automatically. In the latter case, it can be set up and monitored by a bedside intensive care nurse with minimal additional training, another advantage over the current and conventional means of TTM, since it must be used continuously for 24-96 hours. The present invention thus resolves a logistical problem; the need of several trained technicians to provide the service around the clock. Besides, the present invention advantageously decreases the cost and allows its widespread use. In this case, the software of the present invention in the control unit or computer of the present invention will automatically maintain pre-set parameters, make interventions needed, and troubleshoot. The present invention is thus more effective and safer than the existing devices because it can reach a targeted temperature in a shorter time, maintain a stable temperature for a long time, allow rewarming of the body in a programmed way, and decrease the incidence of thromboembolism, all of which improve clinical outcomes and survival for the patients.

In one of the embodiments of the present invention, it provides a device or system or apparatus to deliver targeted temperature management to a patient, comprising: a first whole blood circulation equipment; a circuit of tubes; an extracorporeal cooling unit; and a second whole blood circulation equipment, wherein the device or system or apparatus delivers targeted temperature management by cooling circulating whole blood from a patient extracorporeally to a target temperature as the whole blood circulates through the device or system or apparatus to obtain cooled whole blood at the target temperature that is circulated back into the patient, wherein the targeted temperature management includes cooling, maintaining, and increasing the core temperature of the patient to the target temperature by changing the temperature of the whole blood of the patient being circulated through the device or system or apparatus, wherein the first whole blood circulation equipment is used to draw whole blood from a patient continuously, wherein the first whole blood circulation equipment is selected from a group consisting of a first single lumen catheter, a first metal needle, and a first lumen of a double lumen catheter, wherein the whole blood drawn from the patient circulates at a flow rate in a range of between 50 and 200 milliliters per minute (ml/min) through the circuit of tubes, wherein the extracorporeal cooling unit is selected from a group consisting of one or more heat-exchangers, one or more coiled tubes, and one or more containers containing one or more cooling liquids or cooling fluids or coolants, wherein the cooled whole blood is returned to the patient via the second whole blood circulation equipment, wherein the second whole blood circulation equipment is selected from a group consisting of a second single lumen catheter, a second metal needle, and a second lumen of the double lumen catheter, wherein the first single lumen catheter, the first metal needle, or the first lumen of the double lumen catheter is inserted into a first vein of the patient, and the second single lumen catheter, the second metal needle, or the second lumen of the double lumen catheter is inserted into a second vein of the patient, wherein the first vein and the second vein are different veins of the patient, wherein the first lumen of the double lumen catheter is arterial and referred to as an arterial lumen, and the second lumen of the double lumen catheter is venous and referred to as a venous lumen, wherein the target temperature is a desired low core body temperature for the patient, and it is a pre-determined temperature set by an operator of the device or system or apparatus in a mode selected from a group consisting of manual mode, automatic mode, and semi-automatic mode, and wherein the desired low core temperature is selected from a range of temperatures between 32° C. and 36° C.

In another embodiment of the present invention, it provides the device or system or apparatus to deliver targeted temperature management to a patient as disclosed herein, wherein the device or system or apparatus further comprises: one or more peristaltic pumps; one or more dials to control and change flow rates across various components of the device or system or apparatus; one or more flow rate sensors; one or more dials to control and change temperatures across various components of the device or system or apparatus; one or more temperature sensors; a digital display unit to display the flow rates and the temperatures across various components of the device or system or apparatus; a control unit; one or more air chambers; and one or more alarms for air, wherein the device or system or apparatus can be operated in a mode selected from a group consisting of manual mode, automatic mode, and semi-automatic mode, wherein the one or more air chambers trap air bubbles out of the whole blood from the patient as the whole blood moves through the circuit of tubes across various components of the device or system or apparatus, wherein the one or more alarms for air detect the presence of air bubbles in the whole blood from the patient as the whole blood moves through the circuit of tubes across various components of the device or system or apparatus, wherein the one or more alarms for air is activated to make a visual or audio signal to alert of the presence of air bubbles in the whole blood inside the circuit of tubes in a scenario selected from a group of scenarios consisting of a first scenario that indicates that air bubbles have escaped the one or more air chambers, a second scenario that indicates that there is a misconnection in the circuit of tubes and air is sucked into the circuit of tubes, and a third scenario that indicates a combination of the first scenario and the second scenario, wherein the control unit comprises a computer with software including an artificial intelligence deep learning software and other mechanisms, wherein the control unit operates the device or system or apparatus by operating the first whole blood circulation equipment, the circuit of tubes, the extracorporeal cooling unit, the second whole blood circulation equipment, the one or more peristaltic pumps, the one or more dials to control and change flow rates, the one or more flow rate sensors; the one or more dials to control and change temperatures, the one or more temperature sensors; the digital display unit, the one or more air chambers, and the one or more alarms for air in the automatic mode, and the semiautomatic mode, wherein the device or system or apparatus is operated in the manual mode by operating the first whole blood circulation equipment, the circuit of tubes, the extracorporeal cooling unit, the second whole blood circulation equipment, the one or more peristaltic pumps, the one or more dials to control and change flow rates, the one or more flow rate sensors, the one or more dials to control and change temperatures, the one or more temperature sensors, the digital display unit, the one or more air chambers, and the one or more alarms for air manually, wherein the software maintains pre-set parameters, makes interventions, and troubleshoots on its own without any human interventions for the operation of the device or system or apparatus, wherein the first whole blood circulation equipment, the circuit of tubes, the extracorporeal cooling unit, the second whole blood circulation equipment, the one or more peristaltic pumps, the one or more dials to control and change flow rates, the one or more flow rate sensors, the one or more dials to control and change temperatures, the one or more temperature sensors, the digital display unit, the control unit, the one or more air chambers, and the one or more alarms for air are the various components of the device or system or apparatus and are in fluid communication with each other, and wherein the digital display unit comprises one or more digital display windows.

In another embodiment of the present invention, it provides the device or system or apparatus to deliver targeted temperature management to a patient as disclosed herein, wherein the device or system or apparatus is operated continuously for a time in a range of between 24 hours and 120 hours for a patient from the time of start of the operation of the device or system or apparatus.

In another embodiment of the present invention, it provides the device or system or apparatus to deliver targeted temperature management to a patient as disclosed herein, wherein the patient is a human selected from a group consisting of a human with spontaneous beating hearts after cardiac arrest, a human with traumatic brain injury, a human with brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS.

The embodiments of the present invention as disclosed herein can be used individually or in any combination(s), as a part of any interventional device or used as a modification to the device. The devices of the present invention can be combined with any interventional medical device or functional device used in clinical settings.

The present invention discloses a new device, apparatus, and system to induce hypothermia and deliver targeted temperature management by extracorporeally cooling whole blood. This device does not have a UVL component, and it is different than an earlier invention, U.S. Pat. No. 17,470,371, which described a device to inactivate pathogens in plasma by extracorporeally treating it with UVL, it included a UVL unit, a component to cool plasma and another one to heat plasma, and it was used to lower a patient's body temperature to 37° C., if the UVL and heat components were shut down. In contrast, the device of the present invention as disclosed herein, is to be used in patients with spontaneous beating hearts post cardiac arrest, traumatic brain injury, after brain surgery with or before rise in intracranial pressure, in hypoxic brain injury in neonates, and with extracorporeal membrane oxygenator (ECMO) for cardiogenic shock, and acute respiratory distress syndrome (ARDS).

As explained earlier, hypothermia is defined as a core temperature of <36° C. Mild, moderate, and deep hypothermia range between 33-36° C., 28-32° C., and <28° C., respectively. Targeted Temperature Management (TTM) consists of modification of the body temperature for therapeutic reasons. It has an induction phase to reach a targeted temperature, usually 1-2 hours (hrs., or h), a maintenance phase to maintain the temperature, usually 1 to a few days, and a rewarming phase to gradually increase the temperature to a chosen target, usually 1 to a few days.

The normal oral cavity temperature is 36.8±0.4° C. with diurnal variations of 0.5° C. The rectal temperature is 0.4° C. higher and reflects the core temperature. The lower esophagus is the most accurate noninvasive measurement, and the measurement via a pulmonary artery catheter is the most accurate.

One of the goals of the present invention, as disclosed herein, is to achieve a targeted core temperature in a short time, which is of critical importance for tissue and patients' survival, and prevent potentially fatal complications, such as thromboembolism known to be caused by conventional methods for delivering targeted temperature management for therapeutic hypothermia in such patients.

Cardiac arrest (CA), traumatic brain injury (TBI), brain surgery, hypoxia, etc., are examples of primary events that cause high mortality or disability in survivors owing to complications from ischemia and inflammation. For example, in CA, the outcome depends on the duration of ischemia, the degree of tissue death caused by the ischemia, and the severity of inflammation that occurs during the post-ischemia phase. In another example, in TBI, the severity of ischemia and inflammation plays a crucial role. Hypothermia or targeted temperature management (TTM) prevents cell death, preserves brain function, attenuates inflammation in post-CA and in TIB, and other such primary events, and prevents hyperthermia that often occurs within 48 hours of the primary event, and, in many patients, it decreases mortality or disability. Conventionally, there are several methods to deliver TTM, including application of icy water to the skin, mattresses with circulating icy water or air, or endovascular catheters cooled by circulating water. However, these methods do not reach a targeted core temperature in a short time, which is of critical importance for tissue and patients' survival, are not effective in maintaining the chosen temperature over many hours or days, or cause potentially fatal complications, i.e., thromboembolism caused by endovascular catheters. The present invention provides a solution to these problems with the conventional methods and devices by providing devices, apparatus, systems, and methods of their use to induce hypothermia and deliver TTM by extracorporeally cooling the whole blood of the patient in a much more effective and efficient manner.

In an embodiment of the present invention, it provides a device or system or apparatus to deliver targeted temperature management to a patient, comprising: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10) for cooling liquids or cooling fluids or coolants; a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20), wherein the circuit of tubes with segments transports the whole blood from and back to the patient or a container during in vitro testing after the whole blood circulates once fully through the device or system or apparatus, wherein the first tube (2), the second tube (3), the third tube (4), the fourth tube (5), the fifth tube (6) have an internal diameter in a range of between 1.5 millimeters (mm) and 2 mm for patients who are adults, and in a range of between 0.3 mm and 0.4 mm for patients who are children, wherein the first tube (2), the second tube (3), the third tube (4), the fourth tube (5), the fifth tube (6) have a wall thickness of 1 mm, wherein the first tube (2), the second tube (3), the third tube (4), the fourth tube (5), the fifth tube (6) have a length in a range of between 10 feet (ft) and 14 ft for patients who are adults, and in a range of between 5 ft and 9 ft for patients who are children, wherein the first tube (2), the second tube (3), the third tube (4), the fourth tube (5), the fifth tube (6) are made of material selected from a group consisting of Poly Vinyl Chloride (PVC), Polydimethylsiloxane (PDMS), Dow Corning and combinations thereof, wherein the fifth tube (6) that transports the whole blood back to the patient (1) has the air and clot trap (7) on or in it, wherein the air and clot trap (7) is 7 centimeters (cm) in length and has a diameter of 1.5 cm, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling liquid or cooling fluid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling liquid or cooling fluid or coolant flow rate sensor to regulate the cooling and heat exchange in the cooling unit (9) between the whole blood and the cooling liquid or cooling fluid or coolant, wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core temperature for the patient, wherein the low core temperature is selected from a range of temperatures between 32° C. and 36° C., and wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS.

In another embodiment of the present invention, providing the device or system or apparatus as disclosed herein, wherein the cooling unit (9) is designed to comprise a coiled shape for the second tube (3) that transports the whole blood inside the cooling unit (9), with a wall thickness of 0.5 mm to 1 mm, an internal diameter of 1.8 mm to 2 mm, an external diameter of 2.3 mm to 2.6 mm, and a length of 3 meters (m) to 4 m, and the cooling unit (9) contains a volume of a cooling liquid, wherein the volume of the cooling liquid inside the cooling unit (9) is changed per the temperature of the whole blood as it is cooled inside the cooling unit (9), wherein the volume of the cooling liquid is decreased when the target temperature is reached, and the cooling liquid is moved to the storage container (10) which is placed adjacent to the cooling unit (9), and wherein the volume of the cooling liquid is increased by moving more cooling liquid from the storage container (10) when the target temperature is lower and the whole blood needs to be cooled, and optionally the flow rate of the cooling fluid inside the cooling unit (9) is decreased to aid the heat exchange with the whole blood to cool it.

In another embodiment of the present invention, providing the device or system or apparatus as disclosed herein, wherein the cooling unit (9) is designed to comprise a heat exchanger to cool the whole blood from the patient passing through the cooling unit (9), wherein the heat exchanger is made of a material selected from a group of materials comprising: stainless steel coated with silicone, aluminum, titanium, low carbon steel, and other materials, wherein the heat exchanger has a plate design.

In another embodiment of the present invention, providing the device or system or apparatus as disclosed herein, wherein the cooling unit (9) is designed to comprise a heat exchanger to cool the whole blood from the patient passing through the cooling unit (9), wherein the heat exchanger is made of a material selected from a group of materials comprising: stainless steel coated with silicone, aluminum, titanium, low carbon steel, and other materials, wherein the heat exchanger has a tubular design, wherein in the tubular design, the heat exchanger comprises two or more sets of two tubes, each set having a first tube for transporting the whole blood and a second tube for transporting a cooling liquid or cooling fluid or coolant, wherein the first tube and the second tube are attached to each other, wherein the cooling fluid or cooling liquid or coolant comprises a mixture of water and propylene glycol, and wherein the cooling fluid or cooling liquid or coolant has a freezing point between 0° C. and −59° C.

In another embodiment of the present invention, providing the device or system or apparatus as disclosed herein, wherein the cooling unit (9) is designed to comprise a heat exchanger to cool the whole blood from the patient passing through the cooling unit (9), wherein the heat exchanger is made of a material selected from a group of materials comprising: stainless steel coated with silicone, aluminum, titanium, low carbon steel, and other materials, wherein the heat exchanger has a tubular design, wherein the heat exchanger in the tubular design is a shell, wherein the shell comprises an outer cylindrical case with several tubes inside the outer cylindrical case with spaces in between the inner tubes, wherein the several tubes transport the whole blood, and the cooling fluid or cooling liquid or coolant circulates in the spaces between the several tubes within the outer cylindrical case of the shell, wherein the several tubes each have a length in a range of between 15 cm and 25 cm, an internal diameter of 2 mm, and are made of medical grade steel, wherein the cooling fluid or cooling liquid or coolant comprises a mixture of water and propylene glycol, and wherein the cooling liquid or coolant has a freezing point between 0° C. and −59° C. The “several tubes” as used herein mean for instance, if the tubes have a 2 mm internal diameter, 2-4 tubes could suffice, whereas if the tubes have a small internal diameter between 1-2 mm, a larger number of tubes may be needed, maybe 3-6, or even larger. A higher number of tubes with small internal diameter will allow to slow the flow and increase the surface area of the tubes in contact with the coolant and the volume of blood in contact with cool tubes' surface.

In another embodiment of the present invention, providing the device or system or apparatus as disclosed herein, wherein the cooling unit (9) is designed to comprise a heat exchanger to cool the whole blood from the patient passing through the cooling unit (9), wherein the heat exchanger includes an outer tube consisting of a set of inner tubes that the outer tube encases, wherein the inner tubes transport the whole blood, and wherein the outer tube transports the cooling fluid or cooling liquid or coolant, and the circulating cooling fluid or cooling liquid or coolant cools the whole blood from the patient through heat exchange, wherein the cooling fluid or cooling liquid or coolant comprises a mixture of water and propylene glycol, and wherein the cooling liquid or coolant has a freezing point between 0° C. and −59° C.

In another embodiment of the present invention, providing the device or system or apparatus as disclosed herein, wherein the cooling unit (9) is designed to comprise a heat exchanger to cool the whole blood from the patient passing through the cooling unit (9), wherein the heat exchanger includes two containers connected by a mixing valve, the first container contains a first liquid which is water heated to a temperature of up to 40° C., the second container contains a second liquid which is the cooling fluid or cooling liquid or coolant and it is a mixture of propylene glycol in water at different percentage ratios ranging from 0% propylene glycol to 100% propylene glycol and the remainder is water by percentage of the total, wherein the mixture in the second container is heated to a temperature of equal to or less than 30° C., wherein the mixing valve controls the flow of the first liquid from the first container and the second liquid from the second container to mix the first liquid into the second liquid which is the cooling fluid or cooling liquid or coolant to reach a temperature that brings the resulting temperature of the cooling fluid or cooling liquid or coolant to the target temperature of the whole blood.

In another embodiment of the present invention, providing the device or system or apparatus as disclosed herein, wherein the control unit with the control panel (17) comprise a computer (21) with an auto-feedback mechanism for precise temperature control to reach a target temperature of the whole blood of the patient during an induction phase, a maintenance phase, and a rewarming phase, and to keep the temperature of the whole blood at ±0.1° C. of the target temperature, wherein the targeted temperature management consists of three phases, the induction phase, the maintenance phase, and the rewarming phase, wherein the control unit with the control panel (17) operates the device or system or apparatus in the automatic mode, and semi-automatic mode, wherein the computer (21) comprises software and programs with deep learning methods including artificial intelligence deep learning methods and other mechanisms, wherein the software and programs operate the device or system or apparatus automatically on the basis of a library containing all possible operational steps, problems and interventions, capabilities to choose and execute and a program to allow many of the operational steps to be activated by voice, and wherein the software and programs maintain pre-set parameters, makes interventions, and troubleshoots on its own without any human interventions for the operation of the device or system or apparatus in the automatic mode, and semi-automatic mode.

In another embodiment of the present invention, providing the device or system or apparatus as disclosed herein, wherein the control unit with the control panel (17) comprise dials or knobs to change the whole blood flow rate and the cooling liquid or cooling fluid or coolant flow rate; dials or knobs to change the whole blood temperature and the cooling liquid or cooling fluid or coolant temperature; alarms and switches for monitoring air in the whole blood; a screen to display characteristics comprising, the blood flow rate and the coolant flow rate, temperatures, venous pressure; on and off switches for the cooling unit pump (13) and the peristaltic pump (16) of the device or system of apparatus.

In another embodiment of the present invention, providing the device or system or apparatus as disclosed herein, wherein the peristaltic pump (16) propels the whole blood after it has reached the target temperature, wherein the target temperature is pre-set, regulated, and changed by the control unit with the control panel (17) by causing a negative pressure backstream to draw the whole blood from the patient, wherein the fourth tube (5) to transport the whole blood through the peristaltic pump (16) is compressed by rollers of the peristaltic pump (16), and wherein the fourth tube (5) preferably has a length of 16 cm, an internal diameter of 2 mm, an outer diameter of 4 mm, and a wall thickness of 1 mm.

In another embodiment of the present invention, providing the device or system or apparatus as disclosed herein, wherein the box or shell (20) has a width of 45 cm, a height of 30 cm, and a depth of 30 cm, and wherein the box or shell (20) is mounted on wheels for easy transportation.

The devices, apparatus, or systems of the present invention, as disclosed herein, have peristaltic pumps, dials to change the flow rates and temperatures, a digital display of flow rates and temperatures, alarms for air in blood, and can be operated manually, semi-automatically, or automatically. In the latter case, it can be set up and monitored by a bedside intensive care nurse with minimal additional training, an advantage since it must be used continuously for 24-96 hours. This will resolve a logistical problem: the need for several trained technicians to provide the service around the clock. Besides, it will decrease the cost and allow its widespread use. In this case, the software will automatically maintain pre-set parameters, make interventions needed, and troubleshoot. This device will be more effective and safer than the existing devices because it will reach a targeted temperature in a shorter time, maintain a stable temperature for a long time, allow rewarming of the body in a programmed way, and decrease the incidence of thromboembolism, all of which will improve outcomes.

In an embodiment of the present invention, it provides a method for delivering targeted temperature management to a patient, the method comprising the steps of: (a) providing a device or system or apparatus to deliver targeted temperature management to a patient, the device or system or apparatus comprising: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10); a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20); (b) inserting the first tube (2) inside a first central vein of the patient to draw the whole blood from the patient and transport it to the cooling unit (9) inside the device or system or apparatus; (c) inserting the fifth tube (6) inside a second central vein of the patient to return the whole blood from the device or system or apparatus to the patient (1) after it cools down to a target temperature; (d) administering Heparin immediately before or with the next step; (e) starting the device or system or apparatus to deliver targeted temperature management to the patient and commencing an induction phase of the target temperature management; (f) changing to and commencing a maintenance phase of the target temperature management; (g) changing to and commencing a rewarming phase of the target temperature management; and (h) stopping the device or system or apparatus, wherein the circuit of tubes with segments transports the whole blood from and back to the patient (1) or a container during in vitro testing after the whole blood circulates once fully through the device or system or apparatus, wherein the first tube (2) is referred to as the arterial segment of the circuit of tubes that transports the whole blood from the patient (1) to the cooling unit (9) inside the device or system or apparatus, wherein the fifth tube (6) is referred to as the venous segment of the circuit of tubes that transports and returns the whole blood after cooling the whole blood to the target temperature in the device or system or apparatus to the patient (1), wherein the fifth tube (6) that transports the whole blood back to the patient (1) has the air and clot trap (7) on or in it, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling fluid or cooling liquid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling fluid or coolant flow rate sensor to regulate the cooling and heat exchange in the cooling unit (9) between the whole blood and the cooling liquid or cooling fluid or coolant; wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core body temperature for the patient, wherein the low core temperature is selected from a range of temperatures between 32° C. and 36° C., wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS, wherein the starting the device or system or apparatus to deliver targeted temperature management to the patient is done in a period in a range of between immediately after an event or trauma when spontaneous heartbeats return to minimize brain damage caused by ischemia, and 5 hours after the event or trauma, to prevent post resuscitation worsening of neurological damage triggered by the inflammatory response, and cerebral edema, wherein the core body temperature of the patient is decreased in the induction phase by the device or system or apparatus at a rate of temperature decrease measured in degrees Centigrade (° C.) per hour, which is in a range of between 2° C. to 2.5° C. per hour and 4° C. to 5° C. per hour, wherein the core body temperature of the patient is increased in the rewarming phase to 37° C. by the device or system or apparatus at a rate of temperature increase measured in ° C. per hour, which is in a range of between 0.25° C. per hour and 0.5° C. per hour in patients post a cardiac arrest event, and in a range of between 0.1° C. per hour and 0.25° C. per hour in patients post a traumatic brain injury event, wherein the targeted temperature management is done for a time in a range of between 24 hours and 120 hours for the patient from the starting in step (e) to the stopping in step (h), wherein the method for delivering targeted temperature management is operated in a mode selected from a group consisting of automatic mode, semi-automatic mode, and manual mode, each mode requiring trained personnel of varying intensity, with increasing training and expertise, progressing from the automatic mode to the semi-automatic mode and then to the manual mode.

In another embodiment of the present invention, providing the method for delivering targeted temperature management to a patient as disclosed herein, wherein the control unit with the control panel (17) comprise a computer (21) with an auto-feedback mechanism for precise temperature control to reach a target temperature of the whole blood of the patient during an induction phase, a maintenance phase, and a rewarming phase, and to keep the temperature of the whole blood at ±0.1° C. of the target temperature, wherein the targeted temperature management consists of three phases, the induction phase, the maintenance phase, and the rewarming phase, wherein the control unit with the control panel (17) operates the device or system or apparatus in the automatic mode, and semi-automatic mode, wherein the computer (21) comprises software and programs with deep learning methods including artificial intelligence deep learning methods and other mechanisms, wherein the software and programs operate the device or system or apparatus automatically on the basis of a library containing all possible operational steps, problems and interventions, capabilities to choose and execute and a program to allow many of the operational steps to be activated by voice, and wherein the software and programs maintain pre-set parameters, makes interventions, and troubleshoots on its own without any human interventions for the operation of the device or system or apparatus in the automatic mode, and semi-automatic mode, wherein the control unit with the control panel (17) comprise dials or knobs to change the whole blood flow rate and the cooling liquid or cooling fluid or coolant flow rate; dials or knobs to change the whole blood temperature and the cooling liquid or cooling fluid or coolant temperature; alarms and switches for monitoring air in the whole blood; a screen to display characteristics comprising, the blood flow rate and the coolant flow rate, temperatures, venous pressure; on and off switches for the cooling unit pump (13) and the peristaltic pump (16) of the device or system of apparatus, wherein the peristaltic pump (16) propels the whole blood after it has reached the target temperature, wherein the target temperature is pre-set, regulated, and changed by the control unit with the control panel (17) by causing a negative pressure backstream to draw the whole blood from the patient, and wherein the fourth tube (5) to transport the whole blood through the peristaltic pump (16) is compressed by rollers of the peristaltic pump (16).

In another embodiment of the present invention, providing the method for delivering targeted temperature management to a patient as disclosed herein, wherein the target temperature is reached in small decrements of temperature measured in ° C. per hour in the induction phase, it is then maintained in the maintenance phase for a time in range of at least between 24 hours and 96 hours, it is then re-set and increased in small increments of temperature measured in ° C. per hour to reach a higher temperature during the rewarming phase till the core body temperature of the patient reaches 37° C., and wherein the temperature is increased when the patient is hemodynamically unstable during the induction phase and the maintenance phase.

In another embodiment of the present invention, providing the method for delivering targeted temperature management to a patient as disclosed herein, wherein the whole blood flow rate during the induction phase is in a range of between 100 milliliters per minute (ml/min) and 300 ml/min.

In another embodiment of the present invention, providing the method for delivering targeted temperature management to a patient as disclosed herein, wherein the whole blood flow rate during both the maintenance phase and the rewarming phase is in a range of between 50 milliliters per minute (ml/min) and 100 ml/min.

In an embodiment of the present invention, it provides a method for treating and/or preventing complications associated with ischemia and inflammation in a patient by delivering targeted temperature management to the patient, the method comprising the steps of: (a) identifying a patient in need of targeted temperature management owing to the complications associated with ischemia and inflammation which are secondary to a primary complication; (b) checking and noting parameters of ischemia, inflammation, and physiological health parameters in the patient during a primary event or within 5 hours of a primary event and before the targeted temperature management delivery; (c) providing a device or system or apparatus to deliver targeted temperature management to the patient, the device or system or apparatus comprising: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10); a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20); (d) inserting the first tube (2) inside a first central vein of the patient to draw the whole blood from the patient and transport it to the cooling unit (9) inside the device or system or apparatus; (e) inserting the fifth tube (6) inside a second central vein of the patient to return the whole blood from the device or system or apparatus to the patient (1) after it cools down to a target temperature; (f) administering Heparin immediately before or with the next step; (g) starting the device or system or apparatus to deliver targeted temperature management to the patient and commencing an induction phase of the target temperature management; (h) changing to and commencing a maintenance phase of the target temperature management; (i) changing to and commencing a rewarming phase of the target temperature management; (j) stopping the device or system or apparatus; and (k) checking for parameters of ischemia, inflammation, and physiological health parameters in the patient following the targeted temperature management delivery, wherein the circuit of tubes with segments transports the whole blood from and back to the patient (1) or a container during in vitro testing after the whole blood circulates once fully through the device or system or apparatus, wherein the first tube (2) is referred to as the arterial segment of the circuit of tubes that transports the whole blood from the patient (1) to the cooling unit (9) inside the device or system or apparatus, wherein the fifth tube (6) is referred to as the venous segment of the circuit of tubes that transports and returns the whole blood after cooling the whole blood to the target temperature in the device or system or apparatus to the patient (1), wherein the fifth tube (6) that transports the whole blood back to the patient (1) has the air and clot trap (7) on or in it, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling fluid or cooling liquid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling fluid or coolant flow rate sensor to regulate the cooling and heat exchange in the cooling unit (9) between the whole blood and the cooling liquid or cooling fluid or coolant; wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core body temperature for the patient, wherein the low core temperature is selected from a range of temperatures between 32° C. and 36° C., wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS, wherein the starting the device or system or apparatus to deliver targeted temperature management to the patient is done in a period in a range of between immediately after an event or trauma when spontaneous heartbeats return to minimize brain damage caused by ischemia, and 5 hours after the event or trauma, to prevent post resuscitation worsening of neurological damage triggered by the inflammatory response, and cerebral edema, wherein the core body temperature of the patient is decreased in the induction phase by the device or system or apparatus at a rate of temperature decrease measured in degrees Centigrade (° C.) per hour, which is in a range of between 2° C. to 2.5° C. per hour and 4° C. to 5° C. per hour, wherein the core body temperature of the patient is increased in the rewarming phase to 37° C. by the device or system or apparatus at a rate of temperature increase measured in ° C. per hour, which is in a range of between 0.25° C. per hour and 0.5° C. per hour in patients post a cardiac arrest event, and in a range of between 0.1° C. per hour and 0.25° C. per hour in patients post a traumatic brain injury event, wherein the targeted temperature management is done for a time in a range of between 24 hours and 120 hours for the patient from the starting in step (e) to the stopping in step (h), wherein the method for delivering targeted temperature management is operated in a mode selected from a group consisting of automatic mode, semi-automatic mode, and manual mode, each mode requiring trained personnel of varying intensity, with increasing training and expertise, progressing from the automatic mode to the semi-automatic mode and then to the manual mode.

In another embodiment of the present invention, providing the method for treating and/or preventing complications associated with ischemia and inflammation in a patient by delivering targeted temperature management to the patient as disclosed herein, wherein the primary event includes a cardiac arrest, a traumatic brain injury, brain surgery, hypoxia, hypoxic brain injury in neonates, extracorporeal membrane oxygenator referred to as ECMO for cardiogenic shock, and acute respiratory distress syndrome referred to as ARDS.

In another embodiment of the present invention, providing the method for treating and/or preventing complications associated with ischemia and inflammation in a patient by delivering targeted temperature management to the patient as disclosed herein, wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS.

In another embodiment of the present invention, providing the method for treating and/or preventing complications associated with ischemia and inflammation in a patient by delivering targeted temperature management to the patient as disclosed herein, wherein the control unit with the control panel (17) comprise a computer (21) with an auto-feedback mechanism for precise temperature control to reach a target temperature of the whole blood of the patient during an induction phase, a maintenance phase, and a rewarming phase, and to keep the temperature of the whole blood at ±0.1° C. of the target temperature, wherein the targeted temperature management consists of three phases, the induction phase, the maintenance phase, and the rewarming phase, wherein the control unit with the control panel (17) operates the device or system or apparatus in the automatic mode, and semi-automatic mode, wherein the computer (21) comprises software and programs with deep learning methods including artificial intelligence deep learning methods and other mechanisms, wherein the software and programs operate the device or system or apparatus automatically on the basis of a library containing all possible operational steps, problems and interventions, capabilities to choose and execute and a program to allow many of the operational steps to be activated by voice, and wherein the software and programs maintain pre-set parameters, makes interventions, and troubleshoots on its own without any human interventions for the operation of the device or system or apparatus in the automatic mode, and semi-automatic mode, wherein the control unit with the control panel (17) comprise dials or knobs to change the whole blood flow rate and the cooling liquid or cooling fluid or coolant flow rate; dials or knobs to change the whole blood temperature and the cooling liquid or cooling fluid or coolant temperature; alarms and switches for monitoring air in the whole blood; a screen to display characteristics comprising, the blood flow rate and the coolant flow rate, temperatures, venous pressure; on and off switches for the cooling unit pump (13) and the peristaltic pump (16) of the device or system of apparatus, wherein the peristaltic pump (16) propels the whole blood after it has reached the target temperature, wherein the target temperature is pre-set, regulated, and changed by the control unit with the control panel (17) by causing a negative pressure backstream to draw the whole blood from the patient, and wherein the fourth tube (5) to transport the whole blood through the peristaltic pump (16) is compressed by rollers of the peristaltic pump (16).

In another embodiment of the present invention, providing the method for treating and/or preventing complications associated with ischemia and inflammation in a patient by delivering targeted temperature management to the patient as disclosed herein, wherein the target temperature is reached in small decrements of temperature measured in ° C. per hour in the induction phase, it is then maintained in the maintenance phase for a time in range of at least between 24 hours and 96 hours, it is then re-set and increased in small increments of temperature measured in ° C. per hour to reach a higher temperature during the rewarming phase till the core body temperature of the patient reaches 37° C., and wherein the temperature is increased when the patient is hemodynamically unstable during the induction phase and the maintenance phase.

In another embodiment of the present invention, providing the method for treating and/or preventing complications associated with ischemia and inflammation in a patient by delivering targeted temperature management to the patient as disclosed herein, wherein the whole blood flow rate during the induction phase is in a range of between 100 milliliters per minute (ml/min) and 300 ml/min.

In another embodiment of the present invention, providing the method for treating and/or preventing complications associated with ischemia and inflammation in a patient by delivering targeted temperature management to the patient as disclosed herein, wherein the whole blood flow rate during both the maintenance phase and the rewarming phase is in a range of between 50 milliliters per minute (ml/min) and 100 ml/min.

In another embodiment of the present invention, providing the method for treating and/or preventing complications associated with ischemia and inflammation of the brain in a patient by delivering targeted temperature management to the patient as disclosed herein, wherein the parameters of ischemia of the brain in a patient includes clinical markers of tissue ischemia and even though there are several clinical markers of tissue ischemia, including physical signs, humoral markers, and EEG changes, the most obvious are coma and history of cardiac arrest and status post-resuscitation, post-brain surgery, and any of the other listed and known causes of brain ischemia.

In another embodiment of the present invention, providing the method for treating and/or preventing complications associated with ischemia and inflammation of the brain in a patient by delivering targeted temperature management to the patient as disclosed herein, wherein the parameters of inflammation of the brain in the patient include, among others, elevated levels of C-reactive protein (CRP), fibrinogen, and leukocyte counts. Also, various inflammatory ratios like neutrophil-to-lymphocyte ratio (NLR), platelet-to-lymphocyte ratio (PLR), and systemic immune-inflammation index (SII) can be elevated.

In the devices of the present invention as disclosed herein, the whole blood that is continuously drawn from a patient via a single lumen catheter or metal needle or one lumen of a double lumen catheter (or arterial lumen), circulates at a flow rate of 50-200 ml/min, through a circuit of tubes and an extracorporeal cooling unit (9) that could be a heat exchanger or a container with a cooling fluid and the cooled blood is returned to the patient via another single lumen catheter or metal needle, both inserted in a different vein, or the other lumen of a double lumen catheter (or venous lumen).

The circuit of tubes with segments in the various embodiments of the present invention, as disclosed herein, comprise separate segments of tubes, the first of the tubes (2) to move the blood from the patient (1) (or a container during in vitro testing) to the cooling device or cooling unit (9), a second tube (3) to circulate or move the whole blood from the patient (1) through the cooling unit (9), which in one of the embodiments of the present invention is a coiled tube, a third tube (4) to move the whole blood from this to a peristaltic pump (16), a fourth tube (5) to move the whole blood through the peristaltic pump (16), and a fifth tube (6) to move the whole blood from the peristaltic pump (16) to the patient (1) (or a container during in vitro testing). The segments in the circuit of tubes, namely tubes 2, 3, 4, and 6, have an internal diameter of about 2 mm, a wall thickness of about 1 mm, and a length of about 10 ft, are made of material selected from a group consisting of Poly Vinyl Chloride (PVC), Polydimethylsiloxane (PDMS), Dow Corning and combinations thereof, and are commercially available. The segment in the circuit of tubes, i.e., the fifth tube (6) that moves the whole blood from the peristaltic pump (16) back to the patient (1) has an air and clots trap (7), which is 7 cm long, with 1.5 cm diameter, has a filter to trap clots, and an air chamber to trap air in blood. The fifth tube (6) also has a flow rate sensor (18) to sense the flow rate of cooling liquids or cooling fluids or coolants, and an optical sensor for air (8) in the whole blood.

The peristaltic pump (16) in the various embodiments of the present invention, as disclosed herein, propels the whole blood back to the patient (1) and causes negative pressure backstream to draw blood from the patient (1). The fourth tube (5) or segment of the circuit of tubes that is compressed by the peristaltic pump's rollers is made of PDMS, or Dow Corning, it is about 16 cm long, has an internal diameter of about 2 mm, an outer diameter of about 4 mm, and a wall thickness of about 1 mm, and it is commercially available.

The control unit with the control panel (17) in the various embodiments of the present invention, as disclosed herein, has knobs to change the flow rate of the whole blood and the cooling liquids or cooling fluids or coolants, knobs to change the temperature of the whole blood and the cooling liquids or cooling fluids or coolants, alarms and switches for air in the whole blood, a screen to display the flow rates of the whole blood and the cooling liquids or cooling fluids or coolants, temperatures of the whole blood and the cooling liquids or cooling fluids or coolants, venous pressure, on and off switches for the cooling unit pump (13) and peristaltic pump (16). The control unit with the control panel (17) comprises a computer (21).

The computer (21) in the various embodiments of the present invention, as disclosed herein, has a program or software for computerized temperature control with an auto-feedback mechanism for precise temperature control during the induction, maintenance, and rewarming phases, to keep it at ±0.1° C. of the target temperature. The computer (21) also has a program or software with deep learning methods to operate the device automatically based on a library containing all possible operational steps, problems, and interventions, capabilities to choose and execute, and a program to allow many of the operational steps to be activated by voice. It will have access to all data gathered by thermostats, thermometers, flow meters, etc., and the capability to change settings of temperatures, flow rates, stopping and starting the device, etc.

The cooling unit (9) in some of the embodiments of the present invention, as disclosed herein, has a design, where it has a coiled tube (3), made of PDMS, of up to 1 mm wall thickness, an internal diameter of about 2 mm, an external diameter of about 2.6 mm and up to 3 meters (m) in length. The coiled shape could be achieved by wrapping the tube around a cylinder or laying the tube in an acrylic mesh of about 1 mm thickness, which is inside a rigid frame, and giving it an undulating shape, by affixing it with nails to the mesh. The coiled tube (3) is inside a container or shell of the cooling unit (9) filled with a circulating cooling liquid or cooling fluid or coolant of low freezing point. In this design of the cooling unit (9), the volume of the circulating cooling liquid or cooling fluid or coolant could be decreased when the target temperature has been reached and the cooling liquid or cooling fluid or coolant could be moved to an adjacent storage container (10), so a section of the coiled tube (3) is not exposed to the cooling liquid or cooling fluid or coolant. Alternatively, the percentage (%) of the water and cooling liquid or cooling fluid or coolant mixture is changed (for example, more water and less cooling liquid or cooling fluid or coolant), and the reverse is done to increase cooling of the whole blood in the cooling unit (9), when the temperature must be lowered. Alternatively, the flow rate of the cooling liquid or cooling fluid or coolant can be decreased to decrease the heat exchange. However, any type of plastic tube has low thermal conductivity and likely low efficacy to change the whole blood temperature fast. Therefore, other alternative devices are likely to be more useful.

The cooling unit (9) in some of the embodiments of the present invention, as disclosed herein, has another design, where, the cooling unit (9) cools the whole blood by passing it through a heat exchanger (HE) made of stainless steel coated with silicone, aluminum, titanium, low carbon steel, or other material. The cooling unit (9) in the heat exchanger design has a plate format.

The cooling unit (9) in some of the embodiments of the present invention, as disclosed herein, has another design, where, the cooling unit (9) is a heat exchanger with a tubular design, where it has two or more sets of two tubes, in which each set of tubes has one tube to transport the whole blood and the other tube to transport a cooling liquid or cooling fluid or coolant, and both tubes will be attached to each other.

The cooling unit (9) in some of the embodiments of the present invention, as disclosed herein, has another design, where, the cooling unit (9) is a heat exchanger with a tubular design, where it has a shell and inside it several tubes that transports the whole blood and the cooling liquid or cooling fluid or coolant circulates in the space within the shell and the tubes. The “several tubes” as used herein mean for instance, if the tubes have a 2 mm internal diameter, 2-4 tubes could suffice, whereas if the tubes have a small internal diameter between 1-2 mm, a larger number of tubes may be needed, maybe 3-6, or even larger. A higher number of tubes with small internal diameter will allow to slow the flow and increase the surface area of the tubes in contact with the coolant and the volume of blood in contact with cool tubes' surface.

The cooling unit (9) in some of the embodiments of the present invention, as disclosed herein, has another design, where, the cooling unit (9) is a heat exchanger with a tubular design, where the tubes are straight and the whole blood enters the cooling unit (9) at one end and exits it on the other end, or the tubes could be U shaped and the whole blood enters and exits through the same end of the shell. The whole blood is to circulate through the HE at a flow rate of 50-200 ml/min (that is, half of this volume is in each set of tubes). At the same time, the cooling fluid or cooling liquid or coolant, circulates at the same or lower flow rate, in a closed circuit. The cooling liquid or cooling fluid or coolant is propelled by a continuous flow or peristaltic pump referred to as the cooling unit pump (13). All tubes in this design are between 15-25 cm long, but can be shorter, have about 2 mm internal diameter, and are made of medical-grade steel.

The cooling unit (9) in some of the embodiments of the present invention, as disclosed herein, has another design, where it has several tubes to carry the whole blood, and all the tubes are encased in an outer tube through which the cooling liquid or cooling fluid or coolant circulates. The “several tubes” as used herein mean for instance, if the tubes have a 2 mm internal diameter, 2-4 tubes could suffice, whereas if the tubes have a small internal diameter between 1-2 mm, a larger number of tubes may be needed, maybe 3-6, or even larger. A higher number of tubes with small internal diameter will allow to slow the flow and increase the surface area of the tubes in contact with the coolant and the volume of blood in contact with cool tubes' surface.

The cooling unit (9) in some of the embodiments of the present invention, as disclosed herein, has another design, where the cooling method in the cooling unit (9) includes one container with water heated to 40° C. and another container with water with or without propylene glycol at a temperature of 30° C. or lower, and has a mixing valve that allows the flow of cold or hot water or a combination of them to meet the pre-set required cooling fluid temperature, which is then circulated through the cooling unit (9) for heat exchange with the whole blood.

The cooling liquid or cooling fluid or coolant, used interchangeably in the present invention, as disclosed herein, is a water plus propylene glycol mixture with a freezing point between 0° C. (freezing point of water) and −59° C. (freezing point of propylene glycol). The goal of circulating the whole blood from the patient (1) through the cooling unit (9) of the present invention is to cool the blood by 3-4° C. during one pass of each aliquot of blood through the cooling unit (9) during an exposure of 12-15 seconds. Returning cooled whole blood to the patient (1) after a complete passage through the device of the present invention lowers the core temperature of the patient (1) to a chosen target temperature, for example, 32° C. At a flow rate of 150/min, in ten minutes, 1500 ml of cooled blood will be mixed with the 3.5 liters (L) of warmer blood inside the patient if the patient's blood volume is 5 L. Once the target temperature has been reached, the flow of cooling liquid or cooling fluid or coolant through the cooling unit (9) can automatically stop and restart with each change of +0.1° C. of the core temperature or the flow of coolant decreased or the % of water and coolant changed, or a valve could stop the coolant flow, on and off, for a few seconds during rewarming. To increase the temperature to 37° C. during rewarming, the cooling unit (9) may use a cooling fluid or coolant at the chosen temperature, i.e., 35-37° C. or higher or other hot fluid, or may need a separate heat exchanger.

The cooling unit (9) in various embodiments of the present invention, as disclosed herein, has a thermostat (11) and a first thermometer (12). A cooling unit pump (13) propels the cooling liquid or cooling fluid or coolant from the storage container (10) to the cooling unit (9) and vice versa. A second thermometer (14) and a third thermometer (15) are placed before and after the cooling unit (9), where the whole blood enters and exits the cooling unit (9).

The cooling unit (9) in various embodiments of the present invention, as disclosed herein, where the proper temperature of the cooling fluid or cooling liquid or coolant, the number of sets of tubes in the cooling unit (9) when in its heat exchanger design as described above, and the length of the tubes will require calculations and experimentation to reach optimal efficacy. The thermometers (12, 14, 15) and thermostats (11) are synchronized with the cooling unit pump (13) and with the flow rate sensor for cooling liquids or cooling fluids or coolants (18).

The box or shell (20) in the various embodiments of the present invention, as disclosed herein, is a metal or plastic box or shell to encase all components of the device of the present invention, which is of about 45 cm in width, about 30 cm in depth, and about 30 cm in height or length. The box or shell (20) in the various embodiments of the present invention is mounted on wheels for easy transportation.

In some embodiments of the present invention, the extracorporeal cooling unit (9) has a capacity of between 50 ml to 100 ml when in use for adults and a capacity of 10 ml to 100 ml when in use for children.

In some embodiments of the present invention, the first tube (2), the third tube (4), the fourth tube (5), and the fifth tube (6) have an internal diameter of 1.5 to 2 millimeters (mm) in adults and 0.3 to 0.4 mm in children, and a length of 10 to 14 feet (ft) for adults and 5 to 9 ft in children.

In some embodiments of the present invention, the whole blood circulates through the heat exchanger at a flow rate of between 50 and 200 mL/min which is half the volume of each set of tubes and the cooling fluid or cooling liquid or coolant circulates at either the same flow rate of between 50 and 200 mL/min or a lower flow rate when compared to the flow rate of the whole blood in a close circuit and is propelled through the heat exchanger by a continuous flow or peristaltic pump referred to as the cooling unit pump (13).

In some embodiments of the present invention, the cooling fluid or cooling liquid or coolant comprises water alone, one or more other fluids other than water alone, or a combination or mixture of water and one or more fluids other than water.

In some embodiments of the present invention, the temperature of the cooling liquid or cooling fluid or coolant being pumped or coming into the heat exchanger is increased to 37° C. during rewarming.

In some embodiments of the present invention, the heat exchanger uses a fluid, including water heated to a temperature between 35° C. and 37° C. or higher, and/or other fluid to increase the temperature of the circulating whole blood by heat exchange with the heated cooling liquid or cooling fluid or coolant in the heat exchanger.

In some embodiments of the present invention, the temperature of the cooling liquid or cooling fluid or coolant being pumped or coming into the heat exchanger, the number and sets of tubes in the heat exchanger, the length and internal diameter of the tubes in the heat exchanger, vary with the requirements and are determined by experimentation and by real-time calculations and/or dynamic requirements of treating the patient.

In some embodiments of the present invention, the thermometers and thermostats are synchronized with the cooling unit pump and with a cooling fluid flow rate sensor.

In some embodiments of the present invention, a separate pump, the cooling unit pump (13) moves the cooling liquid or cooling fluid or coolant from the storage container (10) to the cooling unit (9). The temperature of the cooling fluid or cooling liquid or coolant is automatically regulated by a synchronization of a thermostat (11) with the inflow of propylene glycol or hot water. The propylene glycol may come from a separate storage container, propelled by a pump, and the hot water by the pressure of its flow.

The optical sensor (8) for air in the whole blood is needed, like in other extracorporeal blood treatment devices, to detect the presence of air bubbles inside the tube which can occur when an air bubble escapes the air chamber that trap air or if there is a misconnection of tubes and air is sucked into the circuit.

In the various embodiments of the present invention, Heparin is administered immediately before or at the start of the method of delivering targeted temperature management or treating or preventing a patient from the complications of ischemia and inflammation as disclosed in the present invention. The need for anticoagulation decreases or is absent in deep hypothermia, i.e., <35° C., because the blood circulates through the tubes at a low flow rate (<friction), but principally because hypothermia increases bleeding tendencies due to changes in the function of coagulation factors.

In the various embodiments of the present invention, the lowering of the temperature of the whole blood is reached in small increments, i.e., 1° C.-2° C. per hour (° C./h) during the first hour. However, the rate of temperature change is determined by the presence or occurrence of other factors, such as low blood pressure and arrhythmia, while it is applied, and when this occurs, the temperature is decreased in increments of 0.5-1° C./h instead. During the rewarming phase of TTM, small increments of 0.2-0.5° C./h are used. However, again, the rate is determined by the presence or occurrence of other factors, such as blood pressure level.

The device or apparatus or system as disclosed in the present invention can be operated in manual, semi-automatic or automatic modes. Therefore, the personnel will be trained to operate it in each of these different modalities. The automatic mode will still require manual insertion of the catheters to draw blood and manual extraction of the catheter when the treatment is to be discontinued, same day or a few days later. The procedures that can be automated, or controlled by voice command, include changes in the blood flow rate, changes in the target temperature.

The invention will be further explained by the following Examples, which are intended to be purely exemplary of the invention and should not be considered as limiting the invention in any way.

### EXAMPLES

The following example provides exemplary embodiments of the implantable devices and sealing halo units of the present invention.

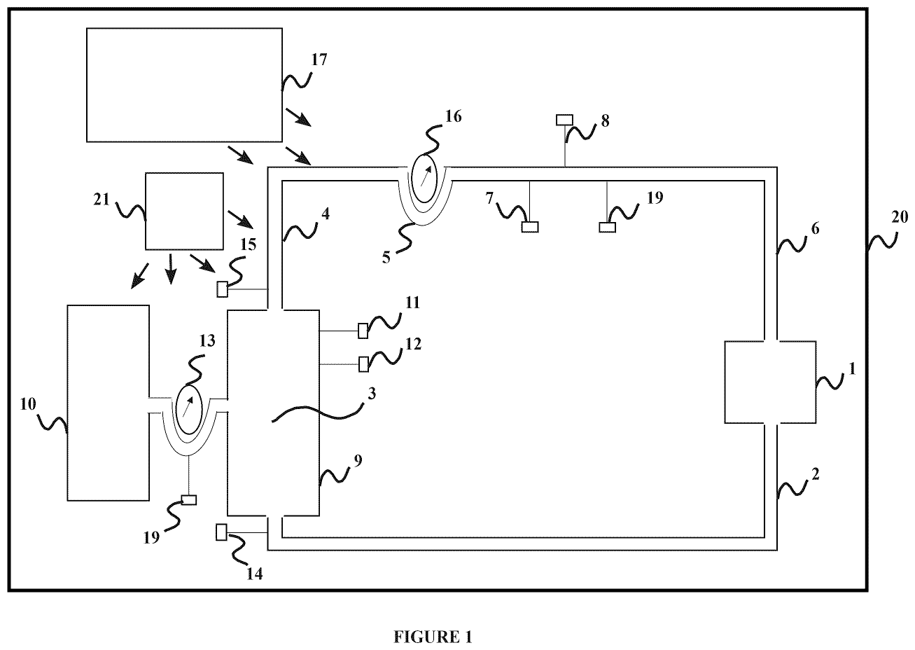

In FIG. 1, an exemplary embodiment of the present invention is illustrated, where the device or system or apparatus to deliver targeted temperature management to a patient, comprises: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10) for cooling liquids or cooling fluids or coolants; a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20), wherein the circuit of tubes with segments transports the whole blood from and back to the patient or a container during in vitro testing after the whole blood circulates once fully through the device or system or apparatus, wherein the first tube (2), the second tube (3), the third tube (4), the fourth tube (5), the fifth tube (6) have an internal diameter in a range of between 1.5 millimeters (mm) and 2 mm for patients who are adults, and in a range of between 0.3 mm and 0.4 mm for patients who are children, wherein the first tube (2), the second tube (3), the third tube (4), the fourth tube (5), the fifth tube (6) have a wall thickness of 1 mm, wherein the first tube (2), the second tube (3), the third tube (4), the fourth tube (5), the fifth tube (6) have a length in a range of between 10 feet (ft) and 14 ft for patients who are adults, and in a range of between 5 ft and 9 ft for patients who are children, wherein the first tube (2), the second tube (3), the third tube (4), the fourth tube (5), the fifth tube (6) are made of material selected from a group consisting of Poly Vinyl Chloride (PVC), Polydimethylsiloxane (PDMS), Dow Corning and combinations thereof, wherein the fifth tube (6) that transports the whole blood back to the patient (1) has the air and clot trap (7) on or in it, wherein the air and clot trap (7) is 7 centimeters (cm) in length and has a diameter of 1.5 cm, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling liquid or cooling fluid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling liquid or cooling fluid or coolant flow rate sensor to regulate the cooling and heat exchange in the cooling unit (9) between the whole blood and the cooling liquid or cooling fluid or coolant, wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core temperature for the patient, wherein the low core temperature is selected from a range of temperatures between 32° C. and 36° C., wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS, wherein the control unit with the control panel (17) comprises a computer (21), wherein the computer (21) has a program or software for computerized temperature control with an auto-feedback mechanism for precise temperature control during the induction, maintenance, and rewarming phases of the targeted temperature management, wherein the computer (21) has a program or software with deep learning methods to operate the device automatically based on a library containing all possible operational steps, problems, and interventions, capabilities to choose and execute, and a program to allow many of the operational steps to be activated by voice, and wherein the computer (21) has access to all data gathered by thermostats, thermometers, flow meters, etc., and the capability to change settings of temperatures, flow rates, stopping and starting the device, etc.

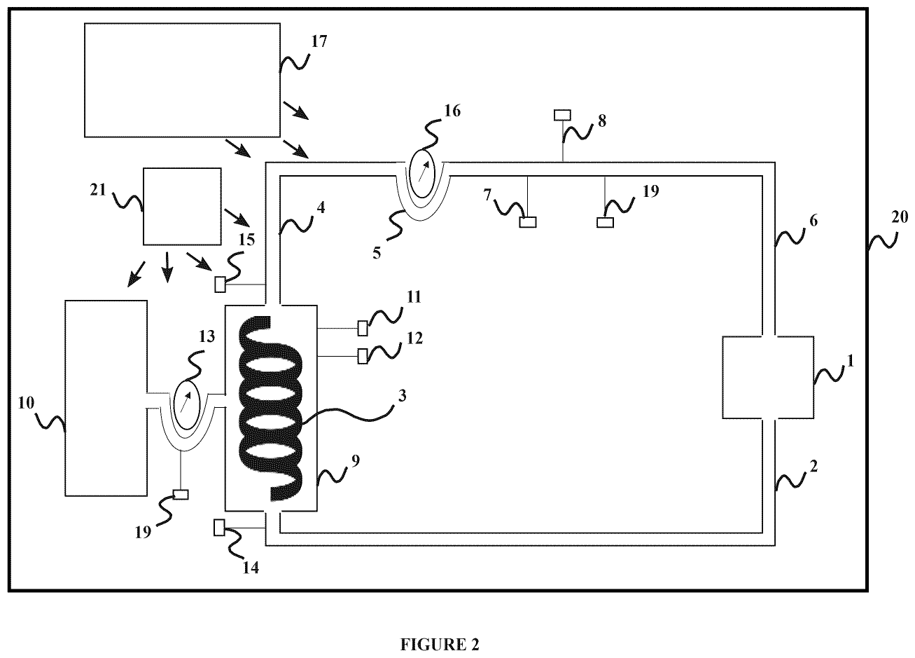

In FIG. 2, an alternative exemplary embodiment of the present invention is illustrated, where the device or system or apparatus to deliver targeted temperature management to a patient, comprises: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10) for cooling liquids or cooling fluids or coolants; a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20), wherein the circuit of tubes with segments transports the whole blood from and back to the patient or a container during in vitro testing after the whole blood circulates once fully through the device or system or apparatus, wherein the first tube (2), the second tube (3), the third tube (4), the fourth tube (5), the fifth tube (6) have an internal diameter in a range of between 1.5 millimeters (mm) and 2 mm for patients who are adults, and in a range of between 0.3 mm and 0.4 mm for patients who are children, wherein the first tube (2), the second tube (3), the third tube (4), the fourth tube (5), the fifth tube (6) have a wall thickness of 1 mm, wherein the first tube (2), the second tube (3), the third tube (4), the fourth tube (5), the fifth tube (6) have a length in a range of between 10 feet (ft) and 14 ft for patients who are adults, and in a range of between 5 ft and 9 ft for patients who are children, wherein the first tube (2), the second tube (3), the third tube (4), the fourth tube (5), the fifth tube (6) are made of material selected from a group consisting of Poly Vinyl Chloride (PVC), Polydimethylsiloxane (PDMS), Dow Corning and combinations thereof, wherein the fifth tube (6) that transports the whole blood back to the patient (1) has the air and clot trap (7) on or in it, wherein the air and clot trap (7) is 7 centimeters (cm) in length and has a diameter of 1.5 cm, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling liquid or cooling fluid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling liquid or cooling fluid or coolant flow rate sensor to regulate the cooling and heat exchange in the cooling unit (9) between the whole blood and the cooling liquid or cooling fluid or coolant, wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core temperature for the patient, wherein the low core temperature is selected from a range of temperatures between 32° C. and 36° C., wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS, wherein the control unit with the control panel (17) comprises a computer (21), wherein the computer (21) has a program or software for computerized temperature control with an auto-feedback mechanism for precise temperature control during the induction, maintenance, and rewarming phases of the targeted temperature management, wherein the computer (21) has a program or software with deep learning methods to operate the device automatically based on a library containing all possible operational steps, problems, and interventions, capabilities to choose and execute, and a program to allow many of the operational steps to be activated by voice, wherein the computer (21) has access to all data gathered by thermostats, thermometers, flow meters, etc., and the capability to change settings of temperatures, flow rates, stopping and starting the device, etc., and wherein the cooling unit (9) is designed to comprise a coiled shape for the second tube (3) that transports the whole blood inside the cooling unit (9), with a wall thickness of 0.5 mm to 1 mm, an internal diameter of 1.8 mm to 2 mm, an external diameter of about 2.3 mm to 2.6 mm, and a length of 3 m to 4 m, and the cooling unit (9) contains a volume of a cooling liquid, wherein the volume of the cooling liquid inside the cooling unit (9) is changed per the temperature of the whole blood as it is cooled inside the cooling unit (9), wherein the volume of the cooling liquid is decreased when the target temperature is reached, and the cooling liquid is moved to the storage container (10) which is placed adjacent to the cooling unit (9), and wherein the volume of the cooling liquid is increased by moving more cooling liquid from the storage container (10) when the target temperature is lower and the whole blood needs to be cooled, and optionally the flow rate of the cooling fluid inside the cooling unit (9) is decreased to aid the heat exchange with the whole blood to cool it.

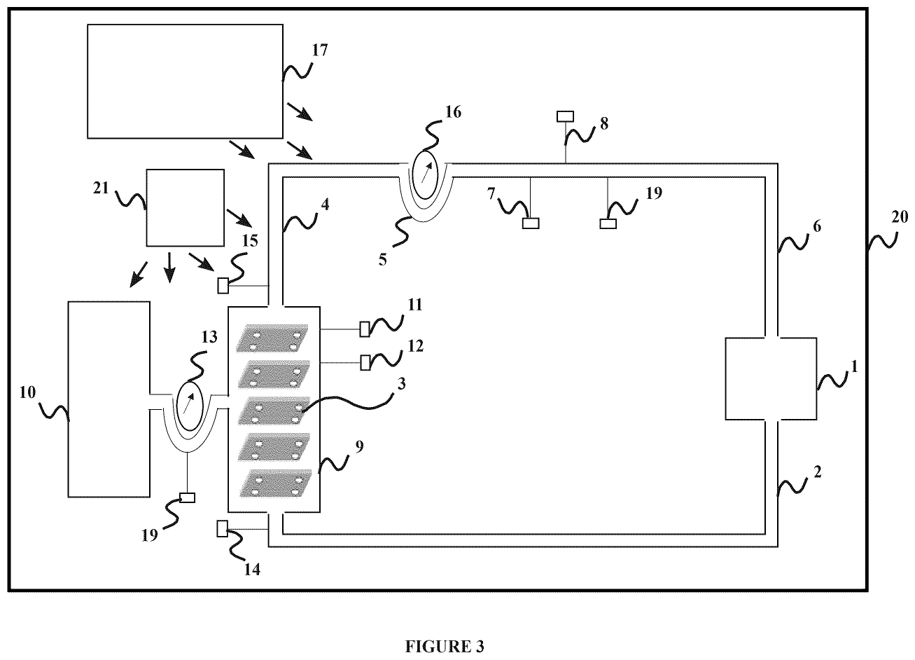

In FIG. 3, an alternative exemplary embodiment of the present invention is illustrated, where the device or system or apparatus to deliver targeted temperature management to a patient, comprises: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10) for cooling liquids or cooling fluids or coolants; a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20), wherein the circuit of tubes with segments transports the whole blood from and back to the patient or a container during in vitro testing after the whole blood circulates once fully through the device or system or apparatus, wherein the first tube (2), the second tube (3), the third tube (4), the fourth tube (5), the fifth tube (6) have an internal diameter in a range of between 1.5 millimeters (mm) and 2 mm for patients who are adults, and in a range of between 0.3 mm and 0.4 mm for patients who are children, wherein the first tube (2), the second tube (3), the third tube (4), the fourth tube (5), the fifth tube (6) have a wall thickness of 1 mm, wherein the first tube (2), the second tube (3), the third tube (4), the fourth tube (5), the fifth tube (6) have a length in a range of between 10 feet (ft) and 14 ft for patients who are adults, and in a range of between 5 ft and 9 ft for patients who are children, wherein the first tube (2), the second tube (3), the third tube (4), the fourth tube (5), the fifth tube (6) are made of material selected from a group consisting of Poly Vinyl Chloride (PVC), Polydimethylsiloxane (PDMS), Dow Corning and combinations thereof, wherein the fifth tube (6) that transports the whole blood back to the patient (1) has the air and clot trap (7) on or in it, wherein the air and clot trap (7) is 7 centimeters (cm) in length and has a diameter of 1.5 cm, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling liquid or cooling fluid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling liquid or cooling fluid or coolant flow rate sensor to regulate the cooling and heat exchange in the cooling unit (9) between the whole blood and the cooling liquid or cooling fluid or coolant, wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core temperature for the patient, wherein the low core temperature is selected from a range of temperatures between 32° C. and 36° C., wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS, wherein the control unit with the control panel (17) comprises a computer (21), wherein the computer (21) has a program or software for computerized temperature control with an auto-feedback mechanism for precise temperature control during the induction, maintenance, and rewarming phases of the targeted temperature management, wherein the computer (21) has a program or software with deep learning methods to operate the device automatically based on a library containing all possible operational steps, problems, and interventions, capabilities to choose and execute, and a program to allow many of the operational steps to be activated by voice, wherein the computer (21) has access to all data gathered by thermostats, thermometers, flow meters, etc., and the capability to change settings of temperatures, flow rates, stopping and starting the device, etc., and wherein the cooling unit (9) is designed to comprise a heat exchanger to cool the whole blood from the patient passing through the cooling unit (9), wherein the heat exchanger is made of a material selected from a group of materials comprising: stainless steel coated with silicone, aluminum, titanium, low carbon steel, and other materials, wherein the heat exchanger has a plate design.

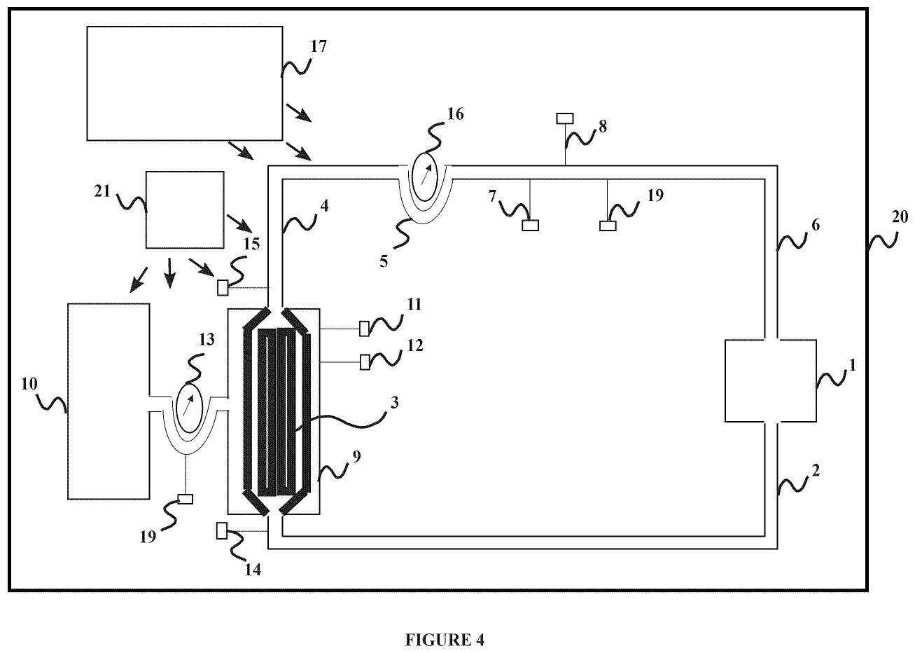

In FIG. 4, an alternative exemplary embodiment of the present invention is illustrated, where the device or system or apparatus to deliver targeted temperature management to a patient, comprises: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10) for cooling liquids or cooling fluids or coolants; a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20), wherein the circuit of tubes with segments transports the whole blood from and back to the patient or a container during in vitro testing after the whole blood circulates once fully through the device or system or apparatus, wherein the first tube (2), the second tube (3), the third tube (4), the fourth tube (5), the fifth tube (6) have an internal diameter in a range of between 1.5 millimeters (mm) and 2 mm for patients who are adults, and in a range of between 0.3 mm and 0.4 mm for patients who are children, wherein the first tube (2), the second tube (3), the third tube (4), the fourth tube (5), the fifth tube (6) have a wall thickness of 1 mm, wherein the first tube (2), the second tube (3), the third tube (4), the fourth tube (5), the fifth tube (6) have a length in a range of between 10 feet (ft) and 14 ft for patients who are adults, and in a range of between 5 ft and 9 ft for patients who are children, wherein the first tube (2), the second tube (3), the third tube (4), the fourth tube (5), the fifth tube (6) are made of material selected from a group consisting of Poly Vinyl Chloride (PVC), Polydimethylsiloxane (PDMS), Dow Corning and combinations thereof, wherein the fifth tube (6) that transports the whole blood back to the patient (1) has the air and clot trap (7) on or in it, wherein the air and clot trap (7) is 7 centimeters (cm) in length and has a diameter of 1.5 cm, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling liquid or cooling fluid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling liquid or cooling fluid or coolant flow rate sensor to regulate the cooling and heat exchange in the cooling unit (9) between the whole blood and the cooling liquid or cooling fluid or coolant, wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core temperature for the patient, wherein the low core temperature is selected from a range of temperatures between 32° C. and 36° C., wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS, wherein the control unit with the control panel (17) comprises a computer (21), wherein the computer (21) has a program or software for computerized temperature control with an auto-feedback mechanism for precise temperature control during the induction, maintenance, and rewarming phases of the targeted temperature management, wherein the computer (21) has a program or software with deep learning methods to operate the device automatically based on a library containing all possible operational steps, problems, and interventions, capabilities to choose and execute, and a program to allow many of the operational steps to be activated by voice, wherein the computer (21) has access to all data gathered by thermostats, thermometers, flow meters, etc., and the capability to change settings of temperatures, flow rates, stopping and starting the device, etc., wherein the cooling unit (9) is designed to comprise a heat exchanger to cool the whole blood from the patient passing through the cooling unit (9), wherein the heat exchanger is made of a material selected from a group of materials comprising: stainless steel coated with silicone, aluminum, titanium, low carbon steel, and other materials, wherein the heat exchanger has a tubular design, wherein in the tubular design, the heat exchanger comprises two or more sets of two tubes, each set having a first tube for transporting the whole blood and a second tube for transporting a cooling liquid or cooling fluid or coolant, wherein the first tube and the second tube are attached to each other, wherein the cooling fluid or cooling liquid or coolant comprises a mixture of water and propylene glycol, and wherein the cooling fluid or cooling liquid or coolant has a freezing point between 0° C. and −59° C.

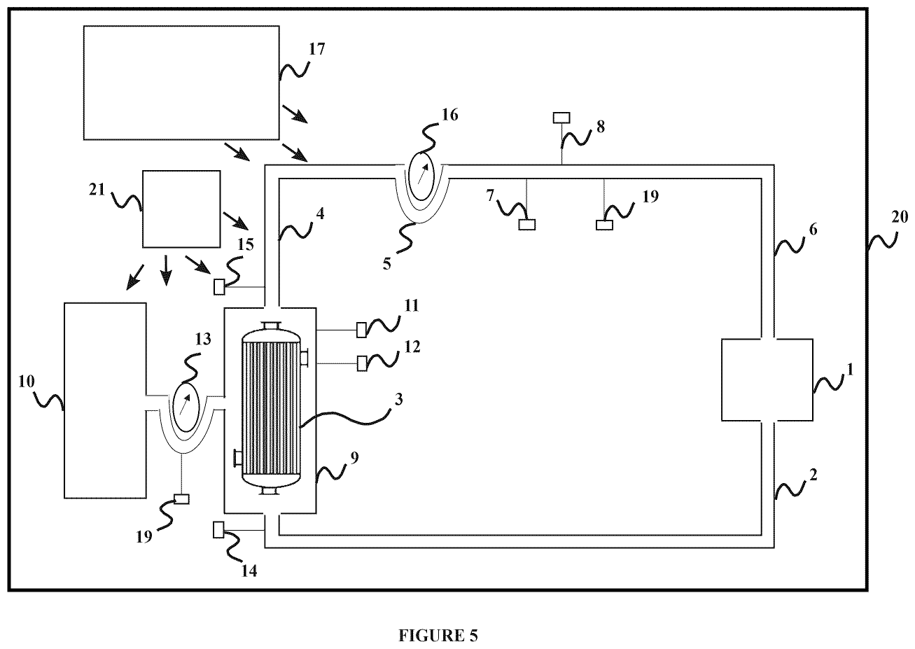

In FIG. 5, an alternative exemplary embodiment of the present invention is illustrated, where the device or system or apparatus to deliver targeted temperature management to a patient, comprises: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10) for cooling liquids or cooling fluids or coolants; a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20), wherein the circuit of tubes with segments transports the whole blood from and back to the patient or a container during in vitro testing after the whole blood circulates once fully through the device or system or apparatus, wherein the first tube (2), the second tube (3), the third tube (4), the fourth tube (5), the fifth tube (6) have an internal diameter in a range of between 1.5 millimeters (mm) and 2 mm for patients who are adults, and in a range of between 0.3 mm and 0.4 mm for patients who are children, wherein the first tube (2), the second tube (3), the third tube (4), the fourth tube (5), the fifth tube (6) have a wall thickness of 1 mm, wherein the first tube (2), the second tube (3), the third tube (4), the fourth tube (5), the fifth tube (6) have a length in a range of between 10 feet (ft) and 14 ft for patients who are adults, and in a range of between 5 ft and 9 ft for patients who are children, wherein the first tube (2), the second tube (3), the third tube (4), the fourth tube (5), the fifth tube (6) are made of material selected from a group consisting of Poly Vinyl Chloride (PVC), Polydimethylsiloxane (PDMS), Dow Corning and combinations thereof, wherein the fifth tube (6) that transports the whole blood back to the patient (1) has the air and clot trap (7) on or in it, wherein the air and clot trap (7) is 7 centimeters (cm) in length and has a diameter of 1.5 cm, wherein the air and clot trap (7) has a filter to trap clots in the whole blood and an air chamber to trap air in the whole blood, wherein the optical air sensor (8) senses air in the whole blood inside the circuit of tubes, including the first tube (2), the second tube (3), the third tube (4), the fourth tube (5), the fifth tube (6), wherein the thermostat (11) and the first thermometer (12) are located on the cooling unit (9), wherein the cooling unit pump (13) moves the cooling liquid or cooling fluid or coolant to and from the cooling unit (9) and the storage container (10), wherein the second thermometer (14) is located before the cooling unit (9), wherein the third thermometer (15) is located after the cooling unit (9), wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, wherein the thermostat (11), the first thermometer (12), the second thermometer (14), and the third thermometer (15) are synchronized with the cooling unit pump (13) and with the cooling liquid or cooling fluid or coolant flow rate sensor to regulate the cooling and heat exchange in the cooling unit (9) between the whole blood and the cooling liquid or cooling fluid or coolant, wherein the device or system or apparatus can be operated in a mode selected from a group consisting of manual mode, automatic mode, and semi-automatic mode, wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17), wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other, wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient, wherein the target temperature is a desired low core temperature for the patient, wherein the low core temperature is selected from a range of temperatures between 32° C. and 36° C., wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS, wherein the control unit with the control panel (17) comprises a computer (21), wherein the computer (21) has a program or software for computerized temperature control with an auto-feedback mechanism for precise temperature control during the induction, maintenance, and rewarming phases of the targeted temperature management, wherein the computer (21) has a program or software with deep learning methods to operate the device automatically based on a library containing all possible operational steps, problems, and interventions, capabilities to choose and execute, and a program to allow many of the operational steps to be activated by voice, wherein the computer (21) has access to all data gathered by thermostats, thermometers, flow meters, etc., and the capability to change settings of temperatures, flow rates, stopping and starting the device, etc., wherein the cooling unit (9) is designed to comprise a heat exchanger to cool the whole blood from the patient passing through the cooling unit (9), wherein the heat exchanger is made of a material selected from a group of materials comprising: stainless steel coated with silicone, aluminum, titanium, low carbon steel, and other materials, wherein the heat exchanger has a tubular design, wherein the heat exchanger in the tubular design is a shell, wherein the shell comprises an outer cylindrical case with several tubes inside the outer cylindrical case with spaces in between the inner tubes, wherein the several tubes transport the whole blood, and the cooling fluid or cooling liquid or coolant circulates in the spaces between the several tubes within the outer cylindrical case of the shell, wherein the several tubes each have a length in a range of between 15 cm and 25 cm, an internal diameter of 2 mm, and are made of medical grade steel, wherein the cooling fluid or cooling liquid or coolant comprises a mixture of water and propylene glycol, and wherein the cooling liquid or coolant has a freezing point between 0° C. and −59° C. The “several tubes” as used herein mean for instance, if the tubes have a 2 mm internal diameter, 2-4 tubes could suffice, whereas if the tubes have a small internal diameter between 1-2 mm, a larger number of tubes may be needed, maybe 3-6, or even larger. A higher number of tubes with small internal diameter will allow to slow the flow and increase the surface area of the tubes in contact with the coolant and the volume of blood in contact with cool tubes' surface.

In another exemplary embodiment of the present invention, an exemplary device, apparatus and system is used to decrease the temperature of the aliquot of blood contained in a cooling unit (9) by 3° C. to 4° C. during one pass of 12 seconds through the cooling unit (9), wherein the aliquot of blood is 100 ml, wherein the flow rate of whole blood through the cooling unit (9) is 200 ml/min, wherein the patient has a total whole blood volume of 5 L blood, and this process of cooling whole blood through the device, apparatus or system of the present invention decreases the core temperature between 32° C. to 34° C. in 25 minutes, which is faster than the 1-2 hours it takes to do so by traditional or conventional devices of targeted temperature management in the market.

In another exemplary embodiment of the present invention, when the surface area of the coiled tube (3) in the cooling unit (9) is 2 mm ID (length x diameter x x), it would allow an aliquot of whole blood circulating through the cooling unit (9) to be exposed for 12-15 seconds to the cooling fluid or cooling liquid or coolant, when the whole blood is circulating at a flow rate of 200 ml/min through the cooling unit (9). Further details will be determined by additional preliminary tests. It could be about 188.4 cm² (length 3000 mm×diameter 2 mm×π, which is 3.14=18840 mm² or 188.4 cm²).

The present invention is significant as it provides the only available device to provide TTM (cooling, maintaining, and increasing the core temperature) to patients' post-cardiac arrest (CA), traumatic brain injury (TBI) and post brain surgery by changing the temperature of the patient's blood while circulating it extracorporeally through a cooling unit (9). Even though there are methods to cool the body surface or the blood of these patients, the device of the present invention has several advantages over the existing methods; it will cool the body faster, will maintain a constant targeted body temperature up to a few days, it will gradually increase the temperature back to normal and it will cause less side effects than the current methods, especially thromboembolism which is of common occurrence with existing endovascular devices. By faster induction of hypothermia, the device will decrease further damage to cells caused by ischemia, inflammation, or cerebral edema, and by maintaining a low temperature for 24 hours to 96 hours or even up to 120 hours or 5 days in total, it will prevent and avoid the damage to cells and tissues caused by hyperthermia, which occurs in 33% of patients post a CA event, for example.

There are many benefits of the devices, systems, and methods of the present invention, including: It lowers the core temperature to 32-34° C. in less than one hour, unlike devices applied to the skin, which require >1-2 hours. It causes less shivering than surface cooling. Any of the available double lumen catheters of low price can be used, unlike the endovascular devices that require proprietary and expensive catheters that have a higher risk of thromboembolism in deep veins and the right atrium. The available catheters have a lower risk of thrombosis because: (a) they are shorter than the endovascular ones, and the larger surface of the latter allows many circulating platelets to impact on it, become activated, and form clots. The extrinsic coagulation factor may also become activated on friction/impact. (b) The walls of the proprietary catheters have a lower temperature, which may increase platelets or factor XII activation, whereas the available catheters have a higher temperature because one lumen transports blood at body temperature and the other lumen cools blood. An available catheter can be kept in place for many days without thrombus formation. The need for anticoagulation decreases or is absent because of the above and because the blood circulates through the tubes at a low flow rate (<flow rate <velocity and turbulence and <cells' friction). The treatment can be started within minutes of the patient reaching a hospital, and the shorter time to reach a target core temperature may delay or avoid neuronal death. The endovascular catheters take longer to lower the temperature because they only cool blood that is in contact with the catheter, in veins that often do not have a high blood flow rate, whereas the new device cools a larger volume of blood because the pump's negative pressure “suctions” blood into the catheter. Since it can be operated automatically, each patient's intensive care nurse can set it up and monitor it after minimal training, an immense advantage since it must be used continuously for 24-96 hrs. This will decrease the cost of care and resolve the logistical problem of needing several shifts of technicians to treat one patient/day.

The above-mentioned embodiments can be used individually or in any combination(s), as a part of any inflatable device or used as a modification to the device. These devices can be any inflatable medical device for treating blockages or functional non-medical devices that can be used in other industries such as the oil industry.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed is:

1. A device or system or apparatus to deliver targeted temperature management to a patient, comprising:
a first whole blood circulation equipment;
a circuit of tubes;
an extracorporeal cooling unit; and
a second whole blood circulation equipment,
wherein the device or system or apparatus delivers targeted temperature management by cooling circulating whole blood from a patient extracorporeally to a target temperature as the whole blood circulates through the device or system or apparatus to obtain cooled whole blood at the target temperature that is circulated back into the patient,
wherein the targeted temperature management includes cooling, maintaining, and increasing the core temperature of the patient to the target temperature by changing the temperature of the whole blood of the patient being circulated through the device or system or apparatus,
wherein the first whole blood circulation equipment is used to draw whole blood from a patient continuously,
wherein the first whole blood circulation equipment is selected from a group consisting of a first single lumen catheter, a first metal needle, and a first lumen of a double lumen catheter,
wherein the whole blood drawn from the patient circulates at a flow rate in a range of between 50 and 200 milliliters per minute (ml/min) through the circuit of tubes, wherein the extracorporeal cooling unit is selected from a group consisting of one or more heat-exchangers, one or more coiled tubes, and one or more containers containing one or more cooling liquids or cooling fluids or coolants,
wherein the cooled whole blood is returned to the patient via the second whole blood circulation equipment,
wherein the second whole blood circulation equipment is selected from a group consisting of a second single lumen catheter, a second metal needle, and a second lumen of the double lumen catheter,
wherein the first single lumen catheter, the first metal needle, or the first lumen of the double lumen catheter is inserted into a first vein of the patient, and the second single lumen catheter, the second metal needle, or the second lumen of the double lumen catheter is inserted into a second vein of the patient,
wherein the first vein and the second vein are different veins of the patient,
wherein the first lumen of the double lumen catheter is arterial and referred to as an arterial lumen, and the second lumen of the double lumen catheter is venous and referred to as a venous lumen,
wherein the target temperature is a desired low core body temperature for the patient, and it is a pre-determined temperature set by an operator of the device or system or apparatus in a mode selected from a group consisting of manual mode, automatic mode, and semi-automatic mode, and
wherein the desired low core temperature is selected from a range of temperatures between 32° C. and 36° C.

2. The device or system or apparatus of claim 1, wherein the device or system or apparatus further comprises:
one or more peristaltic pumps;
one or more dials to control and change flow rates across various components of the device or system or apparatus;
one or more flow rate sensors;
one or more dials to control and change temperatures across various components of the device or system or apparatus;
one or more temperature sensors;
a digital display unit to display the flow rates and the temperatures across various components of the device or system or apparatus;
a control unit;
one or more air chambers; and
one or more alarms for air,
wherein the device or system or apparatus can be operated in a mode selected from a group consisting of manual mode, automatic mode, and semi-automatic mode,
wherein the one or more air chambers trap air bubbles out of the whole blood from the patient as the whole blood moves through the circuit of tubes across various components of the device or system or apparatus,
wherein the one or more alarms for air detect the presence of air bubbles in the whole blood from the patient as the whole blood moves through the circuit of tubes across various components of the device or system or apparatus,
wherein the one or more alarms for air is activated to make a visual or audio signal to alert of the presence of air bubbles in the whole blood inside the circuit of tubes in a scenario selected from a group of scenarios consisting of a first scenario that indicates that air bubbles have escaped the one or more air chambers, a second scenario that indicates that there is a misconnection in the circuit of tubes and air is sucked into the circuit of tubes, and a third scenario that indicates a combination of the first scenario and the second scenario,
wherein the control unit comprises a computer with software including an artificial intelligence deep learning software and other mechanisms,
wherein the control unit operates the device or system or apparatus by operating the first whole blood circulation equipment, the circuit of tubes, the extracorporeal cooling unit, the second whole blood circulation equipment, the one or more peristaltic pumps, the one or more dials to control and change flow rates, the one or more flow rate sensors; the one or more dials to control and change temperatures, the one or more temperature sensors; the digital display unit, the one or more air chambers, and the one or more alarms for air in the automatic mode, and the semiautomatic mode,
wherein the device or system or apparatus is operated in the manual mode by operating the first whole blood circulation equipment, the circuit of tubes, the extracorporeal cooling unit, the second whole blood circulation equipment, the one or more peristaltic pumps, the one or more dials to control and change flow rates, the one or more flow rate sensors, the one or more dials to control and change temperatures, the one or more temperature sensors, the digital display unit, the one or more air chambers, and the one or more alarms for air manually, wherein the software maintains pre-set parameters, makes interventions, and troubleshoots on its own without any human interventions for the operation of the device or system or apparatus,
wherein the first whole blood circulation equipment, the circuit of tubes, the extracorporeal cooling unit, the second whole blood circulation equipment, the one or more peristaltic pumps, the one or more dials to control and change flow rates, the one or more flow rate sensors, the one or more dials to control and change temperatures, the one or more temperature sensors, the digital display unit, the control unit, the one or more air chambers, and the one or more alarms for air are the various components of the device or system or apparatus and are in fluid communication with each other, and
wherein the digital display unit comprises one or more digital display windows.

3. The device or system or apparatus of claim 1, wherein the device or system or apparatus is operated continuously for a time in a range of between 24 hours and 120 hours for a patient from the time of start of the operation of the device or system or apparatus.

4. The device or system or apparatus of claim 1, wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS.

5. A device or system or apparatus to deliver targeted temperature management to a patient, comprising:
a cooling unit (9);
a peristaltic pump (16);
a circuit of tubes with segments, the segments comprising:
a first tube (2) to transport whole blood from a patient (1) to the cooling unit (9);
a second tube (3) to transport the whole blood inside the cooling unit (9);
a third tube (4) to transport the whole blood from the cooling unit (9) to a peristaltic pump (16);
a fourth tube (5) to transport the whole blood through the peristaltic pump (16);
a fifth tube (6) to transport the whole blood back to the patient (1);
an air and clot trap (7);
an optical air sensor (8);
a storage container (10) for cooling liquids or cooling fluids or coolants;
a thermostat (11);
a first thermometer (12);
a cooling unit pump (13);
a second thermometer (14);
a third thermometer (15);
a control unit with a control panel (17);
a flow rate sensor for cooling liquids or cooling fluids or coolants (18);
a flow rate sensor for whole blood (19); and
a box or shell (20),
wherein the circuit of tubes with segments transports the whole blood from and back to the patient or a container during in vitro testing after the whole blood circulates once fully through the device or system or apparatus,
wherein the first tube (2), the second tube (3), the third tube (4), the fourth tube (5), the fifth tube (6) have an internal diameter in a range of between 1.5 millimeters (mm) and 2 mm for patients who are adults, and in a range of between 0.3 mm and 0.4 mm for patients who are children,
wherein the first tube (2), the second tube (3), the third tube (4), the fourth tube (5), the fifth tube (6) have a wall thickness of about 1 mm,
wherein the first tube (2), the second tube (3), the third tube (4), the fourth tube (5), the fifth tube (6) have a length in a range of between 10 feet (ft) and 14 ft for patients who are adults, and in a range of between 5 ft and 9 ft for patients who are children,
wherein the first tube (2), the second tube (3), the third tube (4), the fourth tube (5), the fifth tube (6) are made of material selected from a group consisting of Poly Vinyl Chloride (PVC), Polydimethylsiloxane (PDMS), Dow Corning and combinations thereof,
wherein the fifth tube (6) that transports the whole blood back to the patient (1) has the air and clot trap (7) on or in it,
wherein the air and clot trap (7) is about 7 centimeters (cm) in length and has a diameter of about 1.5 cm,
wherein the air and clot trap (7) has a filter to trap clots in the whole blood and an air chamber to trap air in the whole blood,
wherein the optical air sensor (8) senses air in the whole blood inside the circuit of tubes, including the first tube (2), the second tube (3), the third tube (4), the fourth tube (5), the fifth tube (6),
wherein the thermostat (11) and the first thermometer (12) are located on the cooling unit (9),
wherein the cooling unit pump (13) moves the cooling liquid or cooling fluid or coolant to and from the cooling unit (9) and the storage container (10),
wherein the second thermometer (14) is located before the cooling unit (9),
wherein the third thermometer (15) is located after the cooling unit (9),
wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure,
wherein the thermostat (11), the first thermometer (12), the second thermometer (14), and the third thermometer (15) are synchronized with the cooling unit pump (13) and with the cooling liquid or cooling fluid or coolant flow rate sensor to regulate the cooling and heat exchange in the cooling unit (9) between the whole blood and the cooling liquid or cooling fluid or coolant,
wherein the device or system or apparatus can be operated in a mode selected from a group consisting of manual mode, automatic mode, and semi-automatic mode,
wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17),
wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other,
wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient,
wherein the target temperature is a desired low core temperature for the patient, wherein the low core temperature is selected from a range of temperatures between 32° C. and 36° C., and
wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS.

6. The device or system or apparatus of claim 5, wherein the cooling unit (9) is designed to comprise a coiled shape for the second tube (3) that transports the whole blood inside the cooling unit (9), with a wall thickness of 0.5 mm to 1 mm, an internal diameter of 1.8 mm to 2 mm, an external diameter of 1.8 mm to 2.6 mm, and a length of 3 meters (m) to 4 m, and the cooling unit (9) contains a volume of a cooling liquid, wherein the volume of the cooling liquid inside the cooling unit (9) is changed per the temperature of the whole blood as it is cooled inside the cooling unit (9), wherein the volume of the cooling liquid is decreased when the target temperature is reached, and the cooling liquid is moved to the storage container (10) which is placed adjacent to the cooling unit (9), and wherein the volume of the cooling liquid is increased by moving more cooling liquid from the storage container (10) when the target temperature is lower and the whole blood needs to be cooled, and optionally the flow rate of the cooling fluid inside the cooling unit (9) is decreased to aid the heat exchange with the whole blood to cool it.

7. The device or system or apparatus of claim 5, wherein the cooling unit (9) is designed to comprise a heat exchanger to cool the whole blood from the patient passing through the cooling unit (9), wherein the heat exchanger is made of a material selected from a group of materials comprising: stainless steel coated with silicone, aluminum, titanium, low carbon steel, and other materials, wherein the heat exchanger has a plate design.

8. The device or system or apparatus of claim 5, wherein the cooling unit (9) is designed to comprise a heat exchanger to cool the whole blood from the patient passing through the cooling unit (9), wherein the heat exchanger is made of a material selected from a group of materials comprising: stainless steel coated with silicone, aluminum, titanium, low carbon steel, and other materials, wherein the heat exchanger has a tubular design, wherein in the tubular design, the heat exchanger comprises two or more sets of two tubes, each set having a first tube for transporting the whole blood and a second tube for transporting a cooling liquid or cooling fluid or coolant, wherein the first tube and the second tube are attached to each other, wherein the cooling fluid or cooling liquid or coolant comprises a mixture of water and propylene glycol, and wherein the cooling fluid or cooling liquid or coolant has a freezing point between 0° C. and −59° C.

9. The device or system or apparatus of claim 5, wherein the cooling unit (9) is designed to comprise a heat exchanger to cool the whole blood from the patient passing through the cooling unit (9), wherein the heat exchanger is made of a material selected from a group of materials comprising: stainless steel coated with silicone, aluminum, titanium, low carbon steel, and other materials, wherein the heat exchanger has a tubular design, wherein the heat exchanger in the tubular design is a shell, wherein the shell comprises an outer cylindrical case with several tubes inside the outer cylindrical case with spaces in between the inner tubes, wherein the several tubes transport the whole blood, and the cooling fluid or cooling liquid or coolant circulates in the spaces between the several tubes within the outer cylindrical case of the shell, wherein the several tubes each have a length in a range of between 15 cm and 25 cm, an internal diameter of about 2 mm, and are made of medical grade steel, wherein the cooling fluid or cooling liquid or coolant comprises a mixture of water and propylene glycol, and wherein the cooling liquid or coolant has a freezing point between 0° C. and −59° C.

10. The device or system or apparatus of claim 5, wherein the cooling unit (9) is designed to comprise a heat exchanger to cool the whole blood from the patient passing through the cooling unit (9), wherein the heat exchanger includes an outer tube consisting of a set of inner tubes that the outer tube encases, wherein the inner tubes transport the whole blood, and wherein the outer tube transports the cooling fluid or cooling liquid or coolant, and the circulating cooling fluid or cooling liquid or coolant cools the whole blood from the patient through heat exchange, wherein the cooling fluid or cooling liquid or coolant comprises a mixture of water and propylene glycol, and wherein the cooling liquid or coolant has a freezing point between 0° C. and −59° C.

11. The device or system or apparatus of claim 5, wherein the cooling unit (9) is designed to comprise a heat exchanger to cool the whole blood from the patient passing through the cooling unit (9), wherein the heat exchanger includes two containers connected by a mixing valve, the first container contains a first liquid which is water heated to a temperature of up to 40° C., the second container contains a second liquid which is the cooling fluid or cooling liquid or coolant and it is a mixture of propylene glycol in water at different percentage ratios ranging from 0% propylene glycol to 100% propylene glycol and the remainder is water by percentage of the total, wherein the mixture in the second container is heated to a temperature of equal to or less than 30° C., wherein the mixing valve controls the flow of the first liquid from the first container and the second liquid from the second container to mix the first liquid into the second liquid which is the cooling fluid or cooling liquid or coolant to reach a temperature that brings the resulting temperature of the cooling fluid or cooling liquid or coolant to the target temperature of the whole blood.

12. The device or system or apparatus of claim 5, wherein the control unit with the control panel (17) comprises a computer (21) with an auto-feedback mechanism for precise temperature control to reach a target temperature of the whole blood of the patient during an induction phase, a maintenance phase, and a rewarming phase, and to keep the temperature of the whole blood at ±0.1° C. of the target temperature, wherein the targeted temperature management consists of three phases, the induction phase, the maintenance phase, and the rewarming phase, wherein the control unit with the control panel (17) operates the device or system or apparatus in the automatic mode, and semi-automatic mode, wherein the computer (21) comprises software and programs with deep learning methods including artificial intelligence deep learning methods and other mechanisms, wherein the software and programs operate the device or system or apparatus automatically on the basis of a library containing all possible operational steps, problems and interventions, capabilities to choose and execute and a program to allow many of the operational steps to be activated by voice, and wherein the software and programs maintain pre-set parameters, makes interventions, and troubleshoots on its own without any human interventions for the operation of the device or system or apparatus in the automatic mode, and semi-automatic mode.

13. The device or system or apparatus of claim 5, wherein the control unit with the control panel (17) comprise dials or knobs to change the whole blood flow rate and the cooling liquid or cooling fluid or coolant flow rate; dials or knobs to change the whole blood temperature and the cooling liquid or cooling fluid or coolant temperature; alarms and switches for monitoring air in the whole blood; a screen to display characteristics comprising, the blood flow rate and the coolant flow rate, temperatures, venous pressure; on and off switches for the cooling unit pump (13) and the peristaltic pump (16) of the device or system of apparatus.

14. The device or system or apparatus of claim 5, wherein the peristaltic pump (16) propels the whole blood after it has reached the target temperature, wherein the target temperature is pre-set, regulated, and changed by the control unit with the control panel (17) by causing a negative pressure backstream to draw the whole blood from the patient, wherein the fourth tube (5) to transport the whole blood through the peristaltic pump (16) is compressed by rollers of the peristaltic pump (16), and wherein the fourth tube (5) preferably has a length of about 16 cm, an internal diameter of about 2 mm, an outer diameter of about 4 mm, and a wall thickness of about 1 mm.

15. The device or system or apparatus of claim 5, wherein the box or shell (20) has a width of about 45 cm, a height of about 30 cm, and a depth of about 30 cm, and wherein the box or shell (20) is mounted on wheels for easy transportation.

16. A method for delivering targeted temperature management to a patient, the method comprising the steps of:
(a) providing a device or system or apparatus to deliver targeted temperature management to a patient, the device or system or apparatus comprising: a cooling unit (9); a peristaltic pump (16); a circuit of tubes with segments, the segments comprising: a first tube (2) to transport whole blood from a patient (1) to the cooling unit (9); a second tube (3) to transport the whole blood inside the cooling unit (9); a third tube (4) to transport the whole blood from the cooling unit (9) to a peristaltic pump (16); a fourth tube (5) to transport the whole blood through the peristaltic pump (16); a fifth tube (6) to transport the whole blood back to the patient (1); an air and clot trap (7); an optical air sensor (8); a storage container (10); a thermostat (11); a first thermometer (12); a cooling unit pump (13); a second thermometer (14); a third thermometer (15); a control unit with a control panel (17); a flow rate sensor for cooling liquids or cooling fluids or coolants (18); a flow rate sensor for whole blood (19); and a box or shell (20);
(b) inserting the first tube (2) inside a first central vein of the patient to draw the whole blood from the patient and transport it to the cooling unit (9) inside the device or system or apparatus;
(c) inserting the fifth tube (6) inside a second central vein of the patient to return the whole blood from the device or system or apparatus to the patient (1) after it cools down to a target temperature;
(d) administering Heparin immediately before or with the next step;
(e) starting the device or system or apparatus to deliver targeted temperature management to the patient and commencing an induction phase of the target temperature management;
(f) changing to and commencing a maintenance phase of the target temperature management;
(g) changing to and commencing a rewarming phase of the target temperature management; and
(h) stopping the device or system or apparatus,
wherein the circuit of tubes with segments transports the whole blood from and back to the patient (1) or a container during in vitro testing after the whole blood circulates once fully through the device or system or apparatus,
wherein the first tube (2) is referred to as the arterial segment of the circuit of tubes that transports the whole blood from the patient (1) to the cooling unit (9) inside the device or system or apparatus,
wherein the fifth tube (6) is referred to as the venous segment of the circuit of tubes that transports and returns the whole blood after cooling the whole blood to the target temperature in the device or system or apparatus to the patient (1), wherein the fifth tube (6) that transports the whole blood back to the patient (1) has the air and clot trap (7) on or in it,
wherein the air and clot trap (7) has a filter to trap clots in the whole blood and an air chamber to trap air in the whole blood,
wherein the optical air sensor (8) senses air in the whole blood inside the circuit of tubes, including the first tube (2), the second tube (3), the third tube (4), the fourth tube (5), the fifth tube (6),
wherein the thermostat (11) and the first thermometer (12) are located on the cooling unit (9),
wherein the cooling unit pump (13) moves the cooling fluid or cooling liquid or coolant to and from the cooling unit (9) and the storage container (10),
wherein the second thermometer (14) is located before the cooling unit (9),
wherein the third thermometer (15) is located after the cooling unit (9),
wherein the control unit with the control panel (17) comprises a digital display unit that comprises one or more digital display windows for monitoring various parameters throughout the device or system or apparatus, the parameter consist of: flow rates, temperature of the whole blood, temperature of the cooling liquid or cooling fluid or coolant, venous pressure, knobs for manual changing of target temperature of the whole blood and the cooling liquid or cooling fluid or cooling fluid or coolant, knobs to stop or start the cooling unit pump (13) and to stop or start the peristaltic pump (16), and alarms including visible and auditory alarms for the presence of air and adverse changes in venous pressure, and alarms including visible and auditory alarms for the presence of air and adverse changes in venous pressure,
wherein the thermostat (11), the first thermometer (12), the second thermometer (14), and the third thermometer (15) are synchronized with the cooling unit pump (13) and with the cooling fluid or coolant flow rate sensor to regulate the cooling and heat exchange in the cooling unit (9) between the whole blood and the cooling liquid or cooling fluid or coolant,
wherein the device or system or apparatus can be operated in a mode selected from a group consisting of manual mode, automatic mode, and semi-automatic mode,
wherein the box or shell (20) encases the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17),
wherein the components of the device or system or apparatus, including the cooling unit (9), the peristaltic pump (16), the circuit of tubes with segments, the segments comprising: the first tube (2), the second tube (3), the third tube (4), the fourth tube (5), the fifth tube (6), the air and clot trap (7), the optical air sensor (8), the storage container (10), the thermostat (11), the first thermometer (12), the cooling unit pump (13), the second thermometer (14), the third thermometer (15), the flow rate sensor for cooling liquids or cooling fluids or coolants (18), the flow rate sensor for whole blood (19), and the control unit with the control panel (17) are in fluid communication with each other,
wherein the device or system or apparatus delivers targeted temperature management by changing the temperature of the whole blood from the patient extracorporeally to a target temperature when the whole blood circulates through the device or system or apparatus to obtain cooled whole blood to be circulated back into the patient,
wherein the target temperature is a desired low core body temperature for the patient,
wherein the low core temperature is selected from a range of temperatures between 32° C. and 36° C.,
wherein the patient is a human selected from a group consisting of a human with spontaneous beating hearts after cardiac arrest, a human with a traumatic brain injury, a human after brain surgery with or before rise in intracranial pressure, a human with hypoxic brain injury in neonates, a human with extracorporeal membrane oxygenator referred to as ECMO for cardiogenic shock, and a human with acute respiratory distress syndrome referred to as ARDS,
wherein the starting the device or system or apparatus to deliver targeted temperature management to the patient is done in a period in a range of between immediately after an event or trauma when spontaneous heartbeats return to minimize brain damage caused by ischemia, and 5 hours after the event or trauma, to prevent post resuscitation worsening of neurological damage triggered by the inflammatory response, and cerebral edema,
wherein the core body temperature of the patient is decreased in the induction phase by the device or system or apparatus at a rate of temperature decrease measured in degrees Centigrade (C) per hour, which is in a range of between 2° C. to 2.5° C. per hour and 4° C. to 5° C. per hour,
wherein the core body temperature of the patient is increased in the rewarming phase to 37° C. by the device or system or apparatus at a rate of temperature increase measured in ° C. per hour, which is in a range of between 0.25° C. per hour and 0.5° C. per hour in patients post a cardiac arrest event, and in a range of between 0.1° C. per hour and 0.25° C. per hour in patients post a traumatic brain injury event,
wherein the targeted temperature management is done for a time in a range of between 24 hours and 120 hours for the patient from the starting in step (e) to the stopping in step (h),
wherein the method for delivering targeted temperature management is operated in a mode selected from a group consisting of automatic mode, semi-automatic mode, and manual mode, each mode requiring trained personnel of varying intensity, with increasing training and expertise, progressing from the automatic mode to the semi-automatic mode and then to the manual mode.

17. The method of claim 16, wherein the control unit with the control panel (17) comprise a computer (21) with an auto-feedback mechanism for precise temperature control to reach a target temperature of the whole blood of the patient during an induction phase, a maintenance phase, and a rewarming phase, and to keep the temperature of the whole blood at ±0.1° C. of the target temperature, wherein the targeted temperature management consists of three phases, the induction phase, the maintenance phase, and the rewarming phase, wherein the control unit with the control panel (17) operates the device or system or apparatus in the automatic mode, and semi-automatic mode, wherein the computer (21) comprises software and programs with deep learning methods including artificial intelligence deep learning methods and other mechanisms, wherein the software and programs operate the device or system or apparatus automatically on the basis of a library containing all possible operational steps, problems and interventions, capabilities to choose and execute and a program to allow many of the operational steps to be activated by voice, and wherein the software and programs maintain pre-set parameters, makes interventions, and troubleshoots on its own without any human interventions for the operation of the device or system or apparatus in the automatic mode, and semi-automatic mode, wherein the control unit with the control panel (17) comprise dials or knobs to change the whole blood flow rate and the cooling liquid or cooling fluid or coolant flow rate; dials or knobs to change the whole blood temperature and the cooling liquid or cooling fluid or coolant temperature; alarms and switches for monitoring air in the whole blood; a screen to display characteristics comprising, the blood flow rate and the coolant flow rate, temperatures, venous pressure; on and off switches for the cooling unit pump (13) and the peristaltic pump (16) of the device or system of apparatus, wherein the peristaltic pump (16) propels the whole blood after it has reached the target temperature, wherein the target temperature is pre-set, regulated, and changed by the control unit with the control panel (17) by causing a negative pressure backstream to draw the whole blood from the patient, and wherein the fourth tube (5) to transport the whole blood through the peristaltic pump (16) is compressed by rollers of the peristaltic pump (16).

18. A method of claim 16, wherein the target temperature is reached in small decrements of temperature measured in ° C. per hour in the induction phase, it is then maintained in the maintenance phase for a time in range of at least between 24 hours and 96 hours, it is then re-set and increased in small increments of temperature measured in ° C. per hour to reach a higher temperature during the rewarming phase till the core body temperature of the patient reaches 37° C., and wherein the temperature is increased when the patient is hemodynamically unstable during the induction phase and the maintenance phase.

19. The method of claim 16, wherein the whole blood flow rate during the induction phase is in a range of between 100 milliliters per minute (ml/min) and 300 ml/min.

20. The method of claim 16, wherein the whole blood flow rate during both the maintenance phase and the rewarming phase is in a range of between 50 milliliters per minute (ml/min) and 100 ml/min.
